(19) United States
(12) Patent Application Publication
(10) Pub. No.: US 2023/0272486 A1
(43) Pub. Date: Aug. 31, 2023

(54) TUMOR FRACTION ESTIMATION USING METHYLATION VARIANTS

(71) Applicant: Grail, LLC, Menlo Park, CA (US)

(72) Inventors: Collin Melton, Menlo Park, CA (US); Archana S. Shenoy, Menlo Park, CA (US); Joerg Bredno, Menlo Park, CA (US); Oliver Claude Venn, San Francisco, CA (US); Konstantin Davydov, Redwood City, CA (US); Matthew H. Larson, San Francisco, CA (US)

(21) Appl. No.: 18/169,834

(22) Filed: Feb. 15, 2023

Related U.S. Application Data
(60) Provisional application No. 63/311,402, filed on Feb. 17, 2022.
(60) Provisional application No. 63/408,412, filed on Sep. 20, 2022.
(60) Provisional application No. 63/432,461, filed on Dec. 14, 2022.
(60) Provisional application No. 63/480,859, filed on Jan. 20, 2023.

(51) Int. Cl. C12Q 1/6886; G16B 20/20; G16B 30/10; G06N 20/00; G16B 40/00; G16H 10/40; G16H 50/20

(52) U.S. Cl. CPC C12Q 1/6886; G16B 20/20; G16B 30/10; G06N 20/00; G16B 40/00; G16H 10/40; G16H 50/20; C12Q 2600/154

(57) ABSTRACT

A computer-implemented method for generating a tumor fraction estimate from a DNA sample of a subject is disclosed. The method may include receiving a dataset of methylation sequence reads from the sample of the subject. The method may also include dividing the dataset into a plurality of variants. The method may further include determining methylation states of the plurality of variants. The method may further include filtering the plurality of variants based on a bank of reference sequence reads to generate a filtered subset of variants. The bank may include reads generated from non-cancer samples and biopsy samples of a plurality of tissues of reference individuals. The counts of the methylation states of variants in the filtered subset are determined and input to a model that is trained based on recurrence rates of the variants in the reference sequence reads. The tumor fraction estimate may be generated by the model.

### CROSS-REFERENCE TO RELATED APPLICATIONS

The present application claims the benefit of and priority to U.S. Provisional Application No. 63/311,402 filed on Feb. 17, 2022, U.S. Provisional Application No. 63/408,412 filed on Sep. 20, 2022, U.S. Provisional Application No. 63/432,461 filed on Dec. 14, 2022, and U.S. Provisional Application No. 63/480,859 filed on Jan. 20, 2023, all of which are hereby incorporated by reference in their entirety.

### FIELD OF ART

This disclosure generally relates to early cancer detection via computing models for predicting tumor fraction from nucleic acid samples.

### BACKGROUND

Cancer is a leading cause of death worldwide. The fatality of cancer is heightened by the fact that cancer is usually detected in latter stages, limiting efficacy of treatment options for long-term survival. Current detection methods generally are cancer type specific, i.e., each cancer type is individually screened for. Each individual screening process is tailored to the cancer type. For example, mammography scans are utilized in breast cancer detection, whereas colonoscopy or fecal tests have helped with colorectal cancer detection. Each varied screening method is generally not cross-applicable to other cancer types. Furthermore, present screening methods are encumbered by low detection rates or high false positive rates. Low detection rates often fail to detect early-stage cancers as the cancers are just developing. A high positive rate misdiagnoses cancer-free individuals as positive for cancer status. As a result, most screening tests are only practical when they are used to test individuals who have a high risk of developing the screened cancer, and they have limited ability to detect cancers in the general population. Novel research has implicated aberrant DNA methylation in many disease processes, including cancer. DNA methylation plays a role in regulating gene expression. Thus, aberrant DNA methylation can create issues in normal gene expression pathways, thereby leading to cancer or other diseases. For example, specific patterns of differentially methylated regions may be useful as molecular markers for various disease states. Nonetheless, even such models face a number of challenges. Early cancer detection is particularly challenging due to the miniscule ratio of tumor cells to non-cancer cells in the subject. The miniscule ratio may be on the order of 1:1000, 1:10,000, or even 1:100,000. This creates a challenge of detecting small amounts of cancer signal amidst healthy signal. Moreover, normal cfDNA may be shed by blood cells which may comprise age-related genetic variations, often resembling cancerous aberrant methylation. These anomalously methylated fragments shed from blood cells can often ostensibly inflate cancer signal.

Further challenges arise post-diagnosis of a patient with cancer. For one, healthcare providers need to understand the status of the cancer to tailor and personalize treatment for the patient. Factors that can be evaluated in the treatment decision-making process may include stage of the cancer, whether the cancer is benign or malignant, whether the cancer is spreading, tissue of origin of the cancer, etc. These factors are typically observed from the traditional screening processes, which generally are cumbersome. For two, during and/or after treatment, healthcare providers rely once more on those traditional screening processes to evaluate efficacy of the treatment. These traditional screening processes can take a toll on the patient. For these reasons, there remains a crucial need in the field to accurately and precisely quantify cancer signal (e.g., tumor fraction) as a valuable post-diagnostic tool.

### SUMMARY

The invention(s) described herein this disclosure provide for improvements to cancer detection and treatment, in particular, providing valuable insights into patient's being screened for cancer. The invention(s) may quantify cancer signal in an individual, which may inform prognosis, staging of the cancer, progression of the cancer, monitoring minimum residual disease (MRD), evaluating treatment efficacy, etc. The invention(s) comprise screening for cancer signal in a cell-free deoxyribonucleic acid (cfDNA) sample of a subject. Such cfDNA samples may comprise hundreds of thousands, if not millions, of cfDNA fragments, thereby resulting in a similar order of sequence reads output by a sequencer, or even a multiple of such order based on a sequencing depth of the sample. Each sequence read relating to cfDNA fragments can vary in length, e.g., up to 250, 300, 350, 400, 450, 500, 550, 600, 650, 700, 750, 800, 850, 900, 950, or 1000 bp in length. These next-generation sequencing techniques greatly increase volume of fragments that can be sequenced and analyzed, thereby enabling such models to identify even miniscule amounts of cancer signal in a sample. The invention(s) are capable of screening for cancer generally, or for a plurality of cancer types from a single sample. This improves over conventional screening methods tailored per cancer type by providing a single comprehensive screening that is capable of screening a variety of cancer types from a single cfDNA sample.

The invention(s) implement computer models to identify and quantify the cancer signal based on the shed cfDNA from tumor cells. The computer models may identify the cancer signal from cfDNA fragments including aberrant methylation signatures from sequence reads for cfDNA. The computer models may identify anomalously methylated fragments by building a database of counts of methylation patterns from a healthy population (i.e., subjects without prior diagnosis of disease and/or cancer). The computer models may utilize the database to determine whether a methylation pattern relating to a cfDNA fragment from a test sample is anomalously methylated. The computer models may apply statistical methods to determine the likelihood of observing a fragment in normal subjects (even with fragments having methylation patterns not yet observed in the database). As such, the computer models are capable of identifying the aberrant methylation patterns, the proverbial needles in the haystack.

From the aberrant methylation patterns, the computer models may implement a trained cancer classifier to featurize a sample's aberrant methylation patterns and to generate a cancer prediction. The cancer prediction may be a binary prediction and/or a multiclass prediction. The binary prediction may be a likelihood of presence of cancer. The multiclass prediction may be a likelihood of a particular cancer type. Training a cancer classifier capable of screening between a plurality of cancer types enables medical care professionals to utilize a single comprehensive screening rather than multiple disparate screenings.

The computer models may further train probabilistic models for one or more cancer types to determine a tissue of origin of the anomalously methylated fragments. Each probabilistic model may input a methylation pattern and output a likelihood that the methylation pattern is from a particular cancer type (or more generally disease type). The computer models may featurize methylation patterns that are above threshold likelihoods to further quantify cancer signal in an individual. The ability to quantify cancer signal based on shed fragments from tumor cells improves upon conventional screening methodologies in that conventional methodologies predominantly relied on visual observations by a healthcare professional. Such visual observations lead to subjectivity and human error. This quantification can be practically applied to predict a stage of the cancer, which can inform treatment options available for an individual.

In other example applications, quantification of cancer signal can inform a combination of prognosis, assessment of progression of the cancer, and evaluating treatment efficacy. For example, in informing progression of cancer, the quantification of cancer signal may indicate decreasing tumors, benign tumors, highly aggressive tumors, and/or anything in between. Quantification of cancer signal may also be used in staging of the cancer, e.g., low signal may refer to Stage I, or high signal may refer to a latter stage (e.g., Stage III or IV). Further, the quantification of cancer signal may indicate spreading of a tumor to other tissues in the body. For example, the invention(s) may initially identify cancer signal present in a first tissue of the subject, but then may later identify cancer signal present in a different tissue of the subject, implicating a spreading tumor. In addition, quantification of cancer signal before and during (or after) treatment may inform treatment efficacy in the individual. For example, if prior to treatment, the invention(s) predict cancer signal to be at a first level, but during and/or after treatment, the invention(s) predict cancer signal at a different level, then the difference in levels may implicate efficacy of the treatment. A healthcare professional armed with that knowledge can make better-informed decisions in the subject's treatment, e.g., whether to continue a current treatment when cancer signal is decreasing or to switch treatments if cancer signal remains the same or increases.

A computer-implemented method is disclosed for generating a tumor fraction estimate from a cell-free deoxyribonucleic acid (cfDNA) sample of a subject, the computer-implemented method comprising: receiving a dataset of methylation sequence reads from the cfDNA sample of the subject; dividing the dataset into a plurality of variants, wherein each variant comprises a methylation pattern over one or more CpG sites; filtering the plurality of variants based on a bank of reference sequence reads to generate a filtered subset of variants, the bank comprising reads generated from non-cancer cfDNA samples and biopsy samples of a plurality of tissues of reference individuals; determining, for each variant in the filtered subset, a count of methylation sequence reads that include the variant; inputting the counts of methylation sequence reads for the variants of the filtered subset to a model that is trained based on recurrence rates of the plurality of variants; and generating, using the model, the tumor fraction prediction of the cfDNA sample.

The computer-implemented method is disclosed, wherein the recurrence rates of the plurality of variants are determined based on the reference sequence reads in the bank.

The computer-implemented method is disclosed, wherein filtering the plurality of variants based on reference sequence reads to generate the filtered subset of variants comprises filtering out one or more variants whose rates of presence in the non-cancer samples exceeds a threshold.

The computer-implemented method is disclosed, wherein a particular recurrence rate of a particular variant corresponds to a rate of observation of the particular variant among the reference sequence reads in the bank.

The computer-implemented method is disclosed, wherein the tumor fraction prediction is a distribution of probability of a fraction of fragments in the cfDNA sample that are tumor derived.

The computer-implemented method is disclosed, wherein the tumor fraction prediction is a fraction of fragments in the cfDNA sample that is tumor derived.

The computer-implemented method is disclosed, wherein the model comprises at least one probabilistic model, the probabilistic model comprising a Poisson distribution for a particular variant, and the Poisson distribution is weighted by the recurrence rate of the particular variant.

The computer-implemented method is disclosed, wherein the model comprises a plurality of probabilistic distributions, each probabilistic distribution corresponding to a particular variant and parameterized based on a site-specific noise rate of the particular variant and per site sequencing depth of the particular variant.

The computer-implemented method is disclosed, wherein each probabilistic distribution corresponding to a particular variant is further parameterized based on at least one of: a depth of the cfDNA sample, a targeted panel pull-down efficiency of the cfDNA sample, and an estimated tumor fraction of the cfDNA sample.

The computer-implemented method is disclosed, wherein a count for each variant of the filtered subset comprises a count of methylation sequence reads of the cfDNA sample that include the methylation pattern over the one or more CpG sites of the variant.

The computer-implemented method is disclosed, wherein a particular variant that comprises a plurality of contiguous CpG sites is encoded by a series of binary values, the series corresponds to the contiguous CpG sites, a first binary value at a particular CpG site represents methylation is observed, and a second binary value at the particular CPG site represents unmethylation is observed.

The computer-implemented method is disclosed, wherein the tumor fraction prediction comprises a plurality of fractions for a subset of tissues.

The computer-implemented method is disclosed, wherein each fraction represents a percentage of fragments of the cfDNA sample that is derived from each tissue of the subset of tissues.

The computer-implemented method is disclosed, wherein the model is a binomial mixture model assuming independence between the variants in the filtered set.

The computer-implemented method is disclosed, wherein the model comprises a plurality of methylation sub-models, each methylation sub-model associated with a variant in the filtered set and parameterized by the recurrence rates of the variant across the subset of tissues and an estimated tumor fraction, wherein each methylation sub-model is configured to calculate a likelihood of observing the count of methylation sequence reads based on the count of methylation sequence reads.

The computer-implemented method is disclosed, wherein the model comprises a weighted sum of methylation sub-models enumerating all possibilities for tissues in the subset turned on or off for the variant, each methylation sub-model comprising a weight representing a likelihood of the possibility and parametrized by the recurrence rates of the tissues switched on.

The computer-implemented method is disclosed, wherein each methylation sub-model is a Poisson distribution.

The computer-implemented method is disclosed, wherein the model generates the tumor fraction prediction by identifying an estimated tumor fraction with maximum likelihood as calculated by the methylation sub-models.

The computer-implemented method is disclosed, wherein the maximum likelihood is determined via a grid search.

The computer-implemented method is disclosed, wherein the subset of tissues is selected for a plurality of tissues.

The computer-implemented method is disclosed, wherein the model generates the tumor fraction prediction by identifying an estimated tumor fraction with maximum likelihood as calculated by the methylation sub-models across two or more subsets of tissues, wherein each subset of tissues is a different combination of tissues selected from the plurality of tissues from the other subsets of tissues.

The computer-implemented method, wherein the subsets of tissues are of one size.

The computer-implemented method is disclosed, wherein the size of the subsets of tissues is selected from: 2, 3, or 4.

The computer-implemented method is disclosed, wherein the model comprises a cancer classifier that generates a prediction of one or more tissues from where the fragments are derived, wherein the subset of tissues includes the one or more tissues predicted by the cancer classifier.

The computer-implemented method is disclosed, wherein the methylation sequence reads in the dataset are generated by a targeted methylation assay.

The computer-implemented method is disclosed, wherein the plurality of tissues of the reference individuals used to generate the reference sequence reads are selected from a group comprising a breast tissue, a thyroid tissue, a lung tissue, a bladder tissue, a cervix tissue, small intestine tissue, a colorectal tissue, an esophagus tissue, a gastric tissue, a tonsil tissue, a liver tissue, an ovary tissue, a fallopian tube tissue, a pancreas tissue, a prostate tissue, a kidney tissue, and a uterus tissue.

The computer-implemented method is disclosed, wherein the tumor fraction estimate is generated without a matched biopsy sample.

The computer-implemented method is disclosed, wherein the cfDNA sample is obtained from a bodily fluid without an invasive biopsy.

The computer-implemented method is disclosed, wherein one or more reference individuals in the bank are determined to have cancer or a type of cancer that is breast cancer, uterine cancer, cervical cancer, ovarian cancer, bladder cancer, urothelial cancer of renal pelvis and ureter, renal cancer other than urothelial, prostate cancer, anorectal cancer, colorectal cancer, squamous cell cancer of esophagus, esophageal cancer other than squamous, gastric cancer, hepatobiliary cancer arising from hepatocytes, hepatobiliary cancer arising from cells other than hepatocytes, pancreatic cancer, human-papillomavirus-associated head and neck cancer, head and neck cancer not associated with human papillomavirus, lung adenocarcinoma, small cell lung cancer, squamous cell lung cancer and lung cancer other than adenocarcinoma or small cell lung cancer, neuroendocrine cancer, melanoma, thyroid cancer, sarcoma, multiple myeloma, lymphoma, or leukemia.

The computer-implemented method is disclosed, wherein the model is a machine-learned model.

The computer-implemented method is disclosed, wherein the machine-learned model is one or more of: a constant model, a binomial model, an independent site model, a neural network model, or a Markov model.

The computer-implemented method is disclosed, wherein the machine-learned model is trained by: for each reference sample in the bank including the non-cancer cfDNA samples and the biopsy samples, identifying, for each variant of the filtered variants, a count of reads that include the variant; determining, for each variant of the filtered variants, a recurrence rate for non-cancer based on the counts of reads for the variant in the non-cancer samples; determining, for each variant of the filtered variants, a recurrence rate for cancer based on the counts of reads for the variant in the biopsy samples; and training the model with the recurrence rates for non-cancer and the recurrence rates for cancer, wherein the model is configured to predict a tumor fraction prediction based on counts of reads for the filtered variants in a given sample.

The computer-implemented method is disclosed, wherein the cfDNA sample is used to perform one or more of: cancer surveillance for a previously diagnosed cancer together; and early cancer screening for a plurality of cancer types.

The computer-implemented method is disclosed, wherein the cfDNA sample of the subject is a liquid biopsy collected after beginning of treatment for cancer, the computer-implemented method further comprising: determining a confidence score of the tumor fraction prediction based on the counts of methylation sequence reads that include the filtered subset of variants; in response to determining that the confidence score is below a confidence threshold, sequencing a tissue sample collected after the beginning of the treatment for the cancer; and receiving a second dataset of methylation sequence reads from the tissue sample of the subject; dividing the second dataset into a second plurality of variants; filtering the second plurality of variants based on the bank of reference sequence reads to generate a second filtered subset of variants; determining, for each variant in the filtered subset, a second count of methylation sequence reads that include the variant; inputting the second counts of methylation sequence reads for the variants of the second filtered subset to the model; and generating, using the model, a second tumor fraction prediction of the tissue sample.

The computer-implemented method is disclosed, further comprising: returning the second tumor fraction prediction and the tumor fraction prediction with the confidence score.

The computer-implemented method is disclosed, wherein the cfDNA sample of the subject is a liquid biopsy sample collected after beginning of treatment for cancer, the computer-implemented method further comprising: determining that the tumor fraction prediction of the liquid biopsy sample is below a threshold signal; in response to determining that the tumor fraction prediction of the liquid biopsy sample is below a threshold signal, sequencing a tissue sample collected after the beginning of the treatment for the cancer; and receiving a second dataset of methylation sequence reads from the tissue sample of the subject; dividing the second dataset into a second plurality of variants; filtering the second plurality of variants based on the bank of reference sequence reads to generate a second filtered subset of variants; determining, for each variant in the filtered subset, a second count of methylation sequence reads that include the variant; inputting the second counts of methylation sequence reads for the variants of the second filtered subset to the model; and generating, using the model, a second tumor fraction prediction of the tissue sample.

The computer-implemented method is disclosed, further comprising: returning the second tumor fraction prediction and the tumor fraction prediction below the threshold signal.

The computer-implemented method is disclosed, further comprising: determining a first fraction for a first tissue that is an apoptotic non-cancerous tissue; determining whether the first fraction for the first tissue is above a threshold; and in response to determining that the first fraction for the first tissue is above the threshold, returning an indication the test subject has higher propensity for uncontrolled inflammation as a side effect to immune checkpoint inhibitors.

The computer-implemented method is disclosed, wherein the cfDNA sample is derived from urine of the subject.

The computer-implemented method is disclosed, wherein the tumor fraction prediction is for bladder cancer, prostate cancer, kidney cancer, or some combination thereof.

The computer-implemented method is disclosed, further comprising: determining a poor prognosis for the subject in response to determining that the tumor fraction prediction of the cfDNA sample is above a threshold.

The computer-implemented method is disclosed, wherein the poor prognosis indicates a disease in the subject is aggressive.

The computer-implemented method is disclosed, wherein the poor prognosis indicates the subject is at higher risk of disease recurrence.

The computer-implemented method is disclosed, further comprising: providing a list of recommended treatments based on the poor prognosis for the subject.

The computer-implemented method is disclosed, wherein the cfDNA sample is collected from the subject after beginning of treatment, the method further comprising: determining a presence of residual disease for the subject in response to determining that the tumor fraction prediction of the cfDNA sample is above a threshold.

The computer-implemented method is disclosed, further comprising: providing a list of recommended treatments based on the presence of residual disease for the subject.

The computer-implemented method is disclosed, wherein the cfDNA sample is collected from the subject after beginning of a treatment for a disease in the subject, the method further comprising: evaluating the treatment based on the tumor fraction prediction.

The computer-implemented method is disclosed, wherein evaluating the treatment comprises: determining the treatment to be effective in response to determining that the tumor fraction prediction of the cfDNA sample collected after beginning the treatment is smaller than an initial tumor fraction prediction of an initial cfDNA sample collected before beginning the treatment.

The computer-implemented method is disclosed, wherein evaluating the treatment comprises: determining the treatment to be ineffective in response to determining that the tumor fraction prediction of the cfDNA sample collected after beginning the treatment is substantially equal to or greater than an initial tumor fraction prediction of an initial cfDNA sample collected before beginning the treatment.

The computer-implemented method is disclosed, further comprising: providing a list of alternative treatments excluding the treatment in response to determining that the treatment is ineffective.

A non-transitory computer readable medium is disclosed and configured to store computer code comprising instructions for generating a tumor fraction estimate from a cell-free deoxyribonucleic acid (cfDNA) sample of a subject, wherein the instructions, when executed by one or more processors, cause the one or more processors to perform any of methods disclosed above.

A system is disclosed comprising: one or more processors; and memory configured to store computer code comprising instructions for generating a tumor fraction estimate from a cell-free deoxyribonucleic acid (cfDNA) sample of a subject, wherein the instructions, when executed by the one or more processors, cause the one or more processors to perform any one of methods disclosed above.

### BRIEF DESCRIPTION OF THE DRAWINGS

FIG. 1 is an exemplary flowchart describing an overall workflow of cancer classification of a sample, according to one or more embodiments.

FIG. 2A illustrates a flowchart of devices for sequencing nucleic acid samples according to one embodiment.

FIG. 2B is block diagram of an analytics system for processing sequence reads, according to various embodiments.

FIG. 3A is a flowchart describing a process of sequencing nucleic acids, according to various embodiments.

FIG. 3B is an illustration of a process to obtain a methylation information and methylation state vectors, according to various embodiments.

FIG. 4A illustrates generation of a data structure for a control group, according to various embodiments.

FIG. 4B illustrates a flowchart describing a process of determining anomalously methylated fragments from a sample, according to various embodiments.

FIG. 5 is an illustration of blocks of a reference genome, according to various embodiments.

FIG. 6 is a flowchart of a method for generating a classifier to predict disease state, according to various embodiments.

FIG. 7 is a conceptual diagram illustrating how a tumor fraction estimation model may be trained using a bank of reference sequence reads obtained from reference individuals, according to various embodiments.

FIG. 8 is an example plot of the number of methylation variants found per participant, according to example implementations.

FIG. 9 is an example plot of the recurrence rate of various methylation variants found in different participants, according to example implementations.

FIG. 10 is a flowchart depicting a process for generating a tumor fraction estimate from a cfDNA sample of a subject, according to some embodiments.

FIG. 11 includes plots of the number of methylation variants per megabase in a targeted methylation panel and a WGBS panel, according to example implementations.

FIG. 12 is a plot that illustrates a mean methylation variant cfDNA allele fraction calibration, according to example implementations.

FIG. 13 provides data comparing the estimated tumor fraction in urine and plasma samples from subjects with or without bladder cancer of any stage, according to example implementations.

FIG. 14 provides data comparing the estimated tumor fraction in urine and plasma samples from subjects with or without stage III or IV prostate cancer, according to example implementations.

FIG. 15 provides data comparing the estimated tumor fraction in urine and plasma samples from subjects with or without kidney cancer, according to example implementations.

### DETAILED DESCRIPTION

Reference will now be made in detail to several embodiments, examples of which are illustrated in the accompanying figures. It is noted that wherever practicable similar or like reference numbers may be used in the figures and may indicate similar or like functionality. It is also noted that the contents of all published materials (patent applications, patents, papers, conference proceedings, and the like) referenced herein are incorporated herein by reference in their entirety.

### I. OVERVIEW

### I.A. Definitions

Unless defined otherwise, all technical and scientific terms used herein have the meaning commonly understood by a person skilled in the art to which this description belongs. As used herein, the following terms have the meanings ascribed to them below.

The term “individual” refers to a human, an animal, or any other multicellular living organism. The term “healthy individual” refers to an individual presumed to not have a cancer or disease.

The term “subject” refers to an individual whose DNA is being analyzed. A subject may be a test subject whose DNA is be evaluated using whole genome sequencing or a targeted panel as described herein to evaluate whether the person has a disease state (e.g., cancer, type of cancer, or cancer tissue of origin). A subject may also be part of a control group known not to have cancer or another disease. A subject may also be part of a cancer or other disease group known to have cancer or another disease. Control and cancer/disease groups may be used to assist in designing or validating the targeted panel.

The term “biological sample” or “sample” refers to a specimen obtained from an individual comprising genetic material of the individual. Examples of biological samples include, but are not limited to, blood, whole blood, plasma, serum, urine, cerebrospinal fluid, fecal, saliva, sweat, tears, pleural fluid, pericardial fluid, or peritoneal fluid of the subject. A biological sample can include any tissue or material derived from a living or dead subject. A biological sample can be a cell-free sample. A biological sample can comprise a nucleic acid (e.g., DNA or RNA) or a fragment thereof. The term “nucleic acid” can refer to deoxyribonucleic acid (DNA), ribonucleic acid (RNA) or any hybrid or fragment thereof. The nucleic acid in the sample can be a cell-free nucleic acid. A sample can be a liquid sample or a solid sample (e.g., a cell or tissue sample). A biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, bronchial lavage, discharge fluid from the nipple, aspiration fluid from different parts of the body (e.g., thyroid, breast), etc. A biological sample can be a stool sample. In various embodiments, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free). A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis.

The term “reference sample” refers to a sample obtained from a subject with a known disease state.

The term “training sample” refers to a sample obtained from a known disease state. Training samples may be applied to probability models to generate features that can be utilized for disease state classification.

The term “test sample” refers to a sample that may have an unknown disease state.

The term “sequence read” refers to a nucleotide sequence read from a sample obtained from an individual. Sequence reads may be generated from nucleic acid fragments in the sample. A sequence read can be a collapsed sequence read generated from a plurality of sequence reads derived from a plurality of amplicons from a single original nucleic acid molecule. In some embodiments, the sequence read can be a deduplicated sequence read. Sequence reads can be obtained through various methods known in the art.

The term “disease state” refers to presence or non-presence of a disease, a type of disease, and/or a disease tissue of origin. For example, in one embodiment, the present disclosure provides methods, systems, and non-transitory computer readable medium for detecting cancer (i.e., presence or absence of cancer), a type of cancer, or a cancer tissue of origin.

The term “tissue of origin” or “TOO” refers to the organ, organ group, body region or cell type from which a disease state may arise or originate. For example, the identification of a tissue of origin or cancer cell type typically allows to identify appropriate next steps to further diagnose, stage, and decide on treatment.

The term “methylation” as used herein refers to a chemical process by which a methyl group is added to a DNA molecule. Two of DNA's four bases, cytosine (“C”) and adenine (“A”) can be methylated. For example, a hydrogen atom on the pyrimidine ring of a cytosine base can be converted to a methyl group, forming 5-methylcytosine. Methylation tends to occur at dinucleotides of cytosine and guanine referred to herein as “CpG sites.” In other instances, methylation may occur at a cytosine not part of a CpG site or at another nucleotide that is not cytosine; however, these are rarer occurrences. In this present disclosure, methylation is discussed in reference to CpG sites for the sake of clarity. However, the principles described herein are equally applicable for the detection of methylation in a non-CpG context, including non-cytosine methylation. For example, Adenine methylation has been observed in bacteria, plant and mammalian DNA, although it has received considerably less attention.

In such embodiments, the wet laboratory assay used to detect methylation may vary from those described herein as well known in the art. Further, the methylation state vectors may contain elements that are generally vectors of sites where methylation has or has not occurred (even if those sites are not CpG sites specifically). With that substitution, the remainder of the processes described herein are the same, and consequently the inventive concepts described herein are applicable to those other forms of methylation.

The term “CpG site” refers to a region of a DNA molecule where a cytosine nucleotide is followed by a guanine nucleotide in the linear sequence of bases along its 5′ to 3′ direction. “CpG” is a shorthand for 5′-C-phosphate-G-3′ that is cytosine and guanine separated by only one phosphate group; phosphate links any two nucleotides together in DNA. Cytosines in CpG dinucleotides can be methylated to form 5-methylcytosine.

The term “methylation site” refers to a single site of a DNA molecule where a methyl group can be added. “CpG” sites are the most common methylation site, but methylation sites are not limited to CpG sites. For example, DNA methylation may occur in cytosines in CHG and CHH, where H is adenine, cytosine or thymine. Cytosine methylation in the form of 5-hydroxymethylcytosine may also assessed (see, e.g., WO 2010/037001 and WO 2011/127136, which are incorporated herein by reference), and features thereof, using the methods and procedures disclosed herein. The term “hypomethylated” or “hypermethylated” refers to a methylation status of a DNA molecule containing multiple CpG sites (e.g., more than 3, 4, 5, 6, 7, 8, 9, 10, etc.) where a high percentage of the CpG sites (e.g., more than 80%, 85%, 90%, or 95%, or any other percentage within the range of 50%-100%) are unmethylated or methylated, respectively.

The term “qualifying methylation pattern” refers to a methylation pattern that is in a predetermined CpG site number range, satisfying one or more selection criteria. In the disclosure, the term “qualifying methylation pattern” is used interchangeably with the term “QMP” unless otherwise specified. In some embodiments, a qualifying methylation pattern corresponds to one or more CpG sites (e.g., a span or interval of CpG sites) indexed to a respective one or more specific sites in a reference genome. For example, where a qualifying methylation pattern is identified in a respective one or more fragments in a plurality of fragments aligned to a reference genome, the qualifying methylation pattern comprises one or more CpG sites, where each respective CpG site comprises a respective methylation state and is indexed to a specific site in a reference genome. Thus, in some such embodiments, a qualifying methylation pattern refers to a specific sequence of methylation states at a specific location in a reference genome that satisfies the one or more selection criteria. A qualifying methylation pattern (e.g., a representation of a respective sequence of methylation states for the qualifying methylation pattern such as “MMMMM” or “UUUUU”) may be identified in a respective one or more fragments in a plurality of fragments aligned to a reference genome, where the respective fragment methylation patterns for the plurality of fragments are represented by an interval map, by matching query methylation patterns to representations of each fragment methylation pattern in each node in the interval map, and determining whether the matched methylation patterns satisfy the one or more selection criteria. In some embodiments, a qualifying methylation pattern does not correspond to either a specific CpG site or a specific location in a reference genome (e.g., if the genomic location of the one or more CpG sites in the qualifying methylation is unknown and/or if the sequence of methylation states in the qualifying methylation pattern occurs at multiple locations throughout a reference genome).

The term “cell free deoxyribonucleic nucleic acid,” “cell free DNA,” or “cfDNA” refers to deoxyribonucleic acid fragments that circulate in bodily fluids such as blood, sweat, urine, or saliva and originate from one or more healthy cells and/or from one or more cancer cells.

The term “circulating tumor DNA” or “ctDNA” refers to deoxyribonucleic acid fragments that originate from tumor cells or other types of cancer cells, which may be released into an individual's bodily fluids such as blood, sweat, urine, or saliva as result of biological processes such as apoptosis or necrosis of dying cells or actively released by viable tumor cells.

The term “tumor fraction” refers to the contributing fraction of tumor material to the cfDNA present in a sample. A tumor fraction estimate may further subdivide contribution amounts from underlying tissues and/or cell types.

The term “methylation variant” refers to a pattern of contiguous CpG sites and their statuses that distinguishes DNA derived from one biological source from another. Further, the term “methylation variant allele fraction” (MVAF) is one type of estimate to measure the proportion of abnormally methylated, tumor-derived cfDNA in a sample.

### I.B. Cancer Prediction Workflow

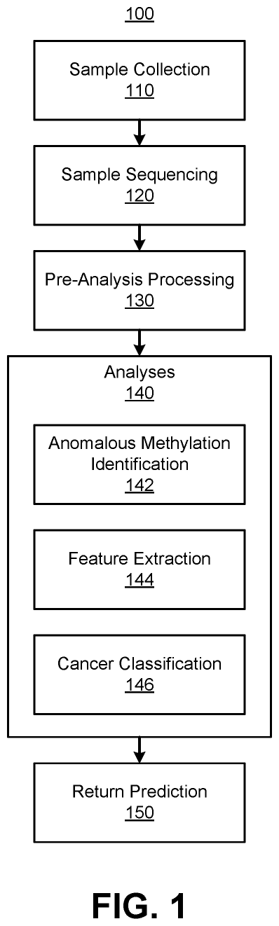

FIG. 1 is an exemplary flowchart describing an overall workflow 100 of cancer classification of a sample, according to one or more embodiments. The workflow 100 is by one or more entities, e.g., including a healthcare provider, a sequencing device, an analytics system, etc. Objectives of the workflow include detecting and/or monitoring cancer in individuals. From a healthcare standpoint, the workflow 100 can serve to supplement other existing cancer diagnostic tools. The workflow 100 may serve to provide early cancer detection and/or routine cancer monitoring to better inform treatment plans for individuals diagnosed with cancer. The overall workflow 100 may include additional/fewer steps than those shown in FIG. 1.

A healthcare provider performs sample collection 110. An individual to undergo cancer classification visits their healthcare provider. The healthcare provider collects the sample for performing cancer classification. Examples of biological samples include, but are not limited to, tissue biopsy, blood, whole blood, plasma, serum, urine, cerebrospinal fluid, fecal, saliva, sweat, tears, pleural fluid, pericardial fluid, or peritoneal fluid of the subject. The sample includes genetic material belonging to the individual, which may be extracted and sequenced for cancer classification. Once the sample is collected, the sample is provided to a sequencing device. Along with the sample, the healthcare provider may collect other information relating to the individual, e.g., biological sex, age, ethnicity, smoking status, any prior diagnoses, etc.

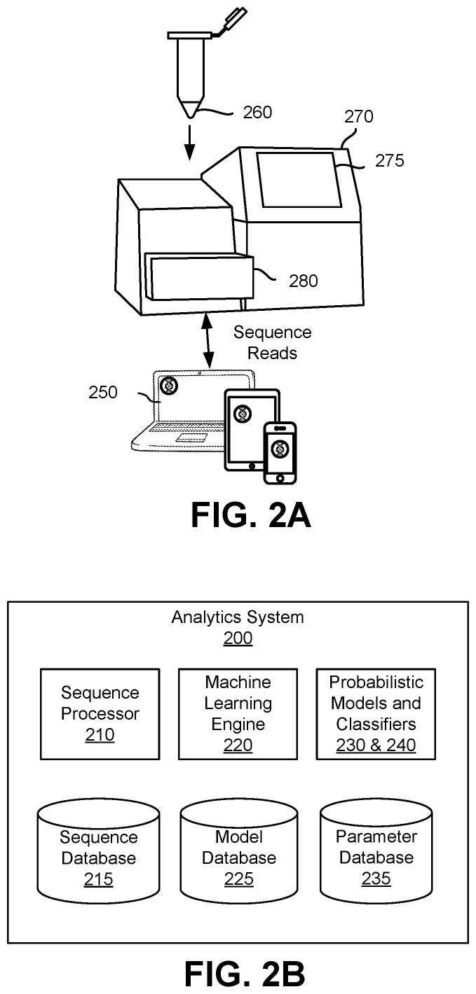

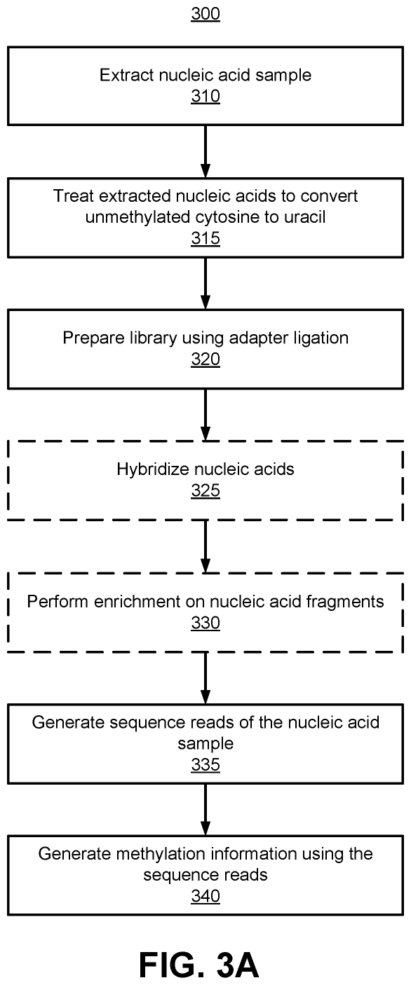

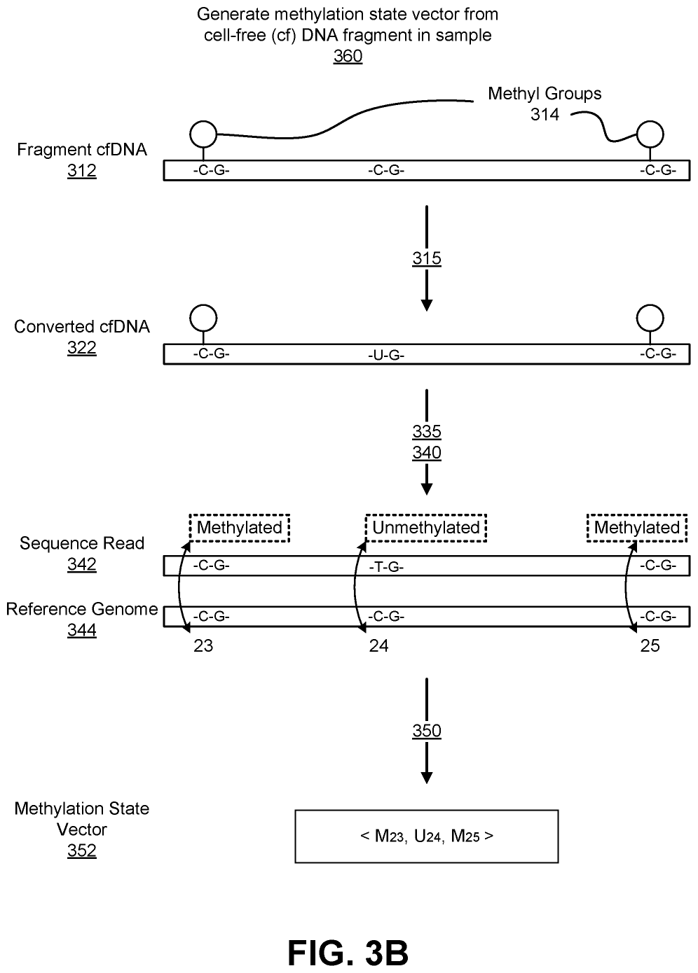

A sequencing device performs sample sequencing 120. A lab clinician may perform one or more processing steps to the sample in preparation of sequencing. Once prepared, the clinician loads the sample in the sequencing device. An example of devices utilizes in sequencing is further described in conjunction with FIGS. 2A & 2B. The sequencing device generally extracts and isolates fragments of nucleic acid that are sequenced to determine a sequence of nucleobases corresponding to the fragments. Sequencing may also include amplification of nucleic material. Different sequencing processes include Sanger sequencing, fragment analysis, and next-generation sequencing. Sequencing may be whole-genome sequencing or targeted sequencing with a target panel. In context of DNA methylation, bisulfite sequencing (e.g., further described in FIGS. 3A & 3B) can determine methylations status through bisulfite conversion of unmethylated cytosines at CpG sites. Sample sequencing 120 yields sequences for a plurality of nucleic acid fragments in the sample. In one or more embodiments, the sequences may include methylation state vectors, wherein each methylation state vector describes the methylation statuses for CpG sites on a fragment.

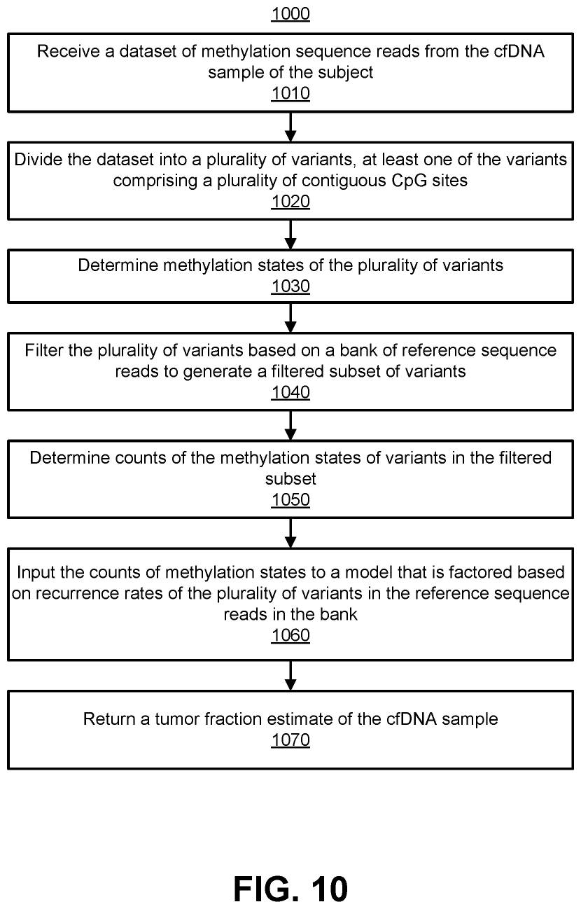

An analytics system performs pre-analysis processing 130. An example analytics system is described in FIG. 10B. Pre-analysis processing 130 may include, but not limited to, de-duplication of sequence reads, determining metrics relating to coverage, determining whether the sample is contaminated, removal of contaminated fragments, calling sequencing error, etc.

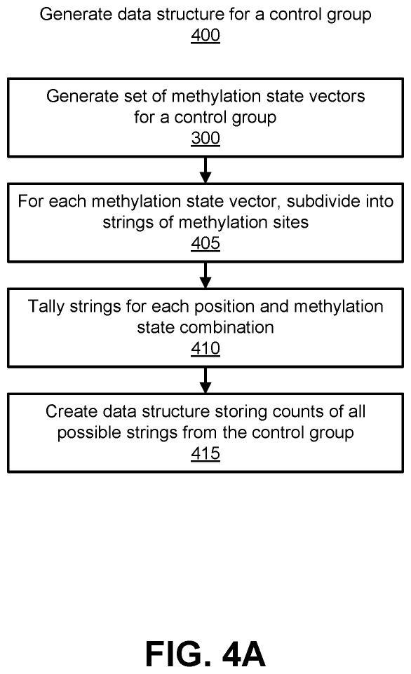

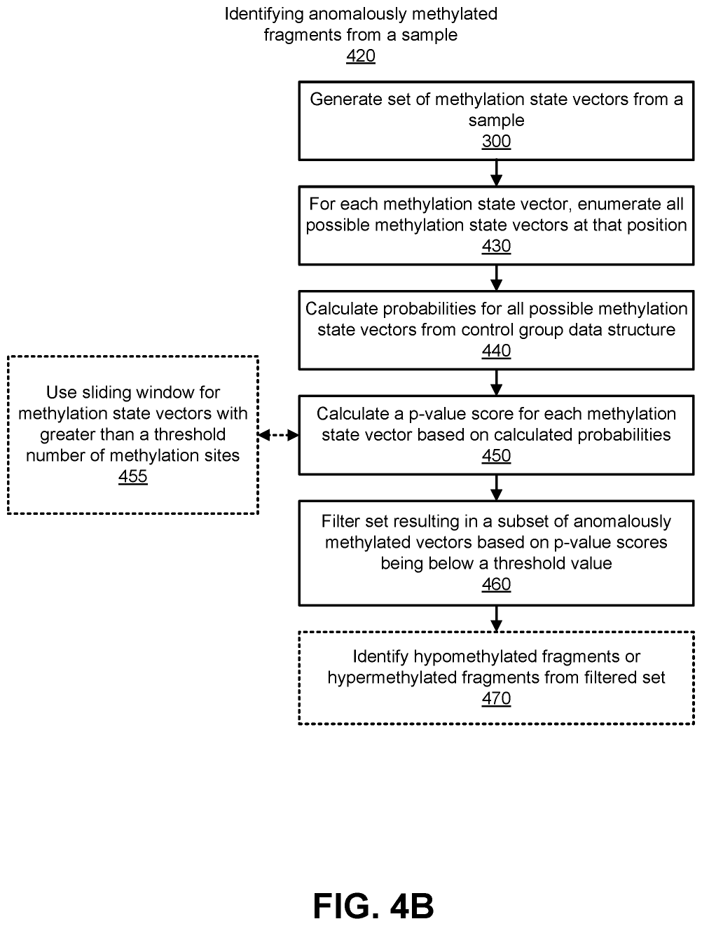

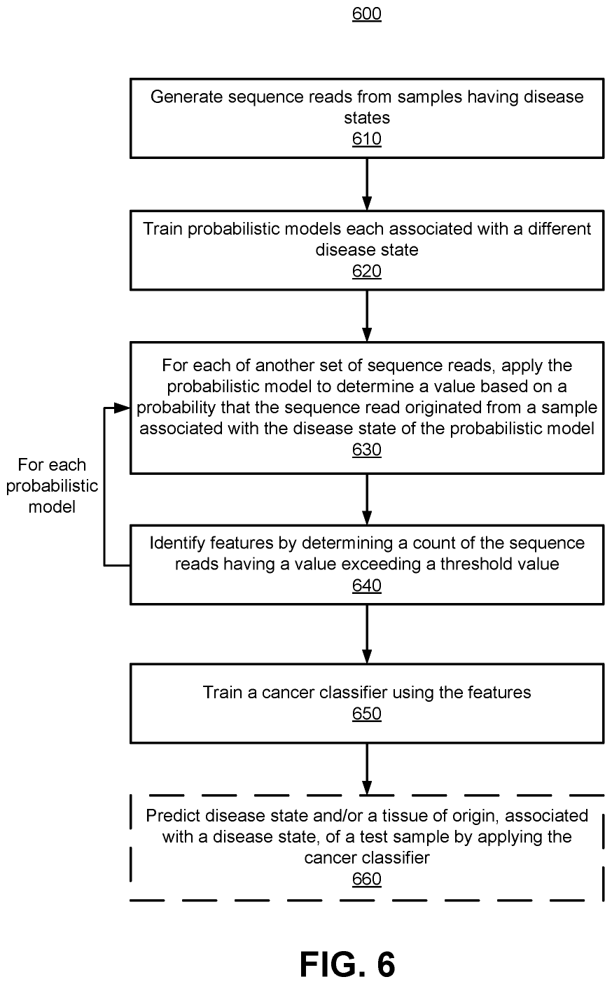

The analytics system performs one or more analyses 140. The analyses are statistical analyses or application of one or more trained models to predict at least a cancer status of the individual from whom the sample is derived. Different genetic features may be evaluated and considered, such as methylation of CpG sites, single nucleotide polymorphisms (SNPs), insertions or deletions (indels), other types of genetic mutation, etc. In context of methylation, analyses 140 may include anomalous methylation identification 142 (e.g., further described in FIGS. 4A & 4B), feature extraction 144 (e.g., further described in FIGS. 6, 7, and 10), and applying a cancer classifier 146 to determine a cancer prediction (e.g., further described in FIGS. 6, 7, and 10). The cancer classifier 146 inputs the extracted features to determine a cancer prediction. The cancer prediction may be a label or a value. The label may indicate a particular cancer state, e.g., binary labels can indicate presence or absence of cancer, multiclass labels can indicate one or more cancer types from a plurality of cancer types that are screened for, a stage of the cancer (e.g., Stage I, II, III, or IV). The value may indicate a likelihood of a particular cancer state, e.g., a likelihood of cancer, and/or a likelihood of a particular cancer type. The prediction 150 may further indicate a quantification of cancer signal, which may include a quantification of one or more particular tissues of origin signals. For example, quantification of cancer signal may be represented as tumor burden calculated based on a percentage of sequence reads determined to be derived from tumor cells.

The analytics system returns the prediction 150 to the healthcare provider. The healthcare provider may establish or adjust a treatment plan based on the cancer prediction. Optimization of treatment is further described in Section VI.C. Treatment.

### I.C. Exemplary Sequencer and Analytics System

FIG. 2A is a flowchart of systems and devices for sequencing nucleic acid samples according to one embodiment. This illustrative flowchart includes devices such as a sequencer 270 and an analytics system 200. The sequencer 270 and the analytics system 200 may work in tandem to perform one or more steps in the processes described herein.

In various embodiments, the sequencer 270 receives an enriched nucleic acid sample 260. As shown in FIG. 2A, the sequencer 270 can include a graphical user interface 275 that enables user interactions with particular tasks (e.g., initiate sequencing or terminate sequencing) as well as one more loading stations 280 for loading a sequencing cartridge including the enriched fragment samples and/or for loading necessary buffers for performing the sequencing assays. Therefore, once a user of the sequencer 270 has provided the necessary reagents and sequencing cartridge to the loading station 280 of the sequencer 270, the user can initiate sequencing by interacting with the graphical user interface 275 of the sequencer 270. Once initiated, the sequencer 270 performs the sequencing and outputs the sequence reads of the enriched fragments from the nucleic acid sample 260.

In some embodiments, the sequencer 270 is communicatively coupled with the analytics system 200. The analytics system 200 includes some number of computing devices used for processing the sequence reads for various applications such as assessing methylation status at one or more CpG sites, variant calling or quality control. The sequencer 270 may provide the sequence reads in a BAM file format to the analytics system 200. The analytics system 200 can be communicatively coupled to the sequencer 270 through a wireless, wired, or a combination of wireless and wired communication technologies. Generally, the analytics system 200 is configured with a processor and non-transitory computer-readable storage medium storing computer instructions that, when executed by the processor, cause the processor to process the sequence reads or to perform one or more steps of any of the methods or processes disclosed herein.

In some embodiments, the sequence reads may be aligned to a reference genome using known methods in the art to determine alignment position information. Alignment position may generally describe a beginning position and an end position of a region in the reference genome that corresponds to a beginning nucleotide based and an end nucleotide base of a given sequence read. Corresponding to methylation sequencing, the alignment position information may be generalized to indicate a first CpG site and a last CpG site included in the sequence read according to the alignment to the reference genome. The alignment position information may further indicate methylation statuses and locations of all CpG sites in a given sequence read. A region in the reference genome may be associated with a gene or a segment of a gene; as such, the analytics system 200 may label a sequence read with one or more genes that align to the sequence read. In one embodiment, fragment length (or size) is determined from the beginning and end positions.

In various embodiments, for example when a paired-end sequencing process is used, a sequence read is comprised of a read pair denoted as R_1 and R_2. For example, the first read R_1 may be sequenced from a first end of a double-stranded DNA (dsDNA) molecule whereas the second read R_2 may be sequenced from the second end of the double-stranded DNA (dsDNA). Therefore, nucleotide base pairs of the first read R_1 and second read R_2 may be aligned consistently (e.g., in opposite orientations) with nucleotide bases of the reference genome. Alignment position information derived from the read pair R_1 and R_2 may include a beginning position in the reference genome that corresponds to an end of a first read (e.g., R_1) and an end position in the reference genome that corresponds to an end of a second read (e.g., R_2). In other words, the beginning position and end position in the reference genome represent the likely location within the reference genome to which the nucleic acid fragment corresponds. In one embodiment, the read pair R_1 and R_2 can be assembled into a fragment, and the fragment used for subsequent analysis and/or classification. An output file having SAM (sequence alignment map) format or BAM (binary) format may be generated and output for further analysis.

Referring now to FIG. 2B, FIG. 2B is a block diagram of an analytics system 200 for processing DNA samples according to one embodiment. The analytics system implements one or more computing devices for use in analyzing DNA samples. The analytics system 200 includes a sequence processor 210, sequence database 215, model database 225, one or more probabilistic models 230 and/or one or more classifiers 240, and parameter database 235. In some embodiments, the analytics system 200 performs one or more steps in the methods or processes disclosed herein.

The sequence processor 210 generates methylation state vectors for fragments from a sample. At each CpG site on a fragment, the sequence processor 210 generates a methylation state vector for each fragment specifying a location of the fragment in the reference genome, a number of CpG sites in the fragment, and the methylation state of each CpG site in the fragment whether methylated, unmethylated, or indeterminate via the process 360 of FIG. 4A. The sequence processor 210 may store methylation state vectors for fragments in the sequence database 215. Data in the sequence database 215 may be organized such that the methylation state vectors from a sample are associated to one another.

Further, multiple different models 230 may be stored in the model database 225 or retrieved for use with test samples. In one example, a model is a trained cancer classifier 240 for determining a cancer prediction for a test sample using a feature vector derived from anomalous fragments. The training and use of the cancer classifier is discussed elsewhere herein. The analytics system 200 may train the one or more models 230 and/or one or more classifiers 240 and store various trained parameters in the parameter database 235. The analytics system 200 stores the models 230 and/or classifiers along with functions in the model database 225.

During inference, the machine learning engine 220 uses the one or more models 230 and/or classifiers 240 to return outputs. The machine learning engine accesses the models 230 and/or classifiers 240 in the model database 225 along with trained parameters from the parameter database 235. According to each model, the machine learning engine 220 receives an appropriate input for the model and calculates an output based on the received input, the parameters, and a function of each model relating the input and the output. In some use cases, the machine learning engine 220 further calculates metrics correlating to a confidence in the calculated outputs from the model. In other use cases, the machine learning engine 220 calculates other intermediary values for use in the model.

### II. ASSAY PROTOCOL

FIG. 3A is a flowchart describing a process 300 of sequencing nucleic acids, according to an embodiment. In some embodiments, the process 300 is performed to generate the sequence reads as part of sample sequencing 120 of the method 100 of FIG. 1.

In step 310, a nucleic acid sample (e.g., DNA or RNA) is extracted from a subject. In the present disclosure, DNA and RNA can be used interchangeably unless otherwise indicated. That is, the embodiments described herein can be applicable to both DNA and RNA types of nucleic acid sequences. However, the examples described herein can focus on DNA for purposes of clarity and explanation. The sample can include nucleic acid molecules derived from any subset of the human genome, including the whole genome. The sample can include blood, plasma, serum, urine, fecal, saliva, other types of bodily fluids, or any combination thereof. In some embodiments, methods for drawing a blood sample (e.g., syringe or finger prick) can be less invasive than procedures for obtaining a tissue biopsy, which can require surgery. The extracted sample can comprise cfDNA and/or ctDNA. If a subject has a disease state, such as cancer, cell free nucleic acids (e.g., cfDNA) in an extracted sample from the subject generally includes detectable level of the nucleic acids that can be used to assess a disease state.

In step 315, the extracted nucleic acids (e.g., including cfDNA fragments) are treated to convert unmethylated cytosines to uracils. In some embodiments, the method 300 uses a bisulfite treatment of the samples which converts the unmethylated cytosines to uracils without converting the methylated cytosines. For example, a commercial kit such as the EZ DNA Methylation™—Gold, EZ DNA Methylation™—Direct or an EZ DNA Methylation™—Lightning kit (available from Zymo Research Corp (Irvine, Calif.)) is used for the bisulfite conversion. In another embodiment, the conversion of unmethylated cytosines to uracils is accomplished using an enzymatic reaction. For example, the conversion can use a commercially available kit for conversion of unmethylated cytosines to uracils, e.g., APOBEC-Seq (NEBiolabs, Ipswich, Mass.).

In step 320, a sequencing library is prepared. In some embodiments, the preparation includes at least two steps. In a first step, a ssDNA adapter is added to the 3′-OH end of a bisulfite-converted ssDNA molecule using a ssDNA ligation reaction. In some embodiments, the ssDNA ligation reaction uses CircLigase II (Epicentre) to ligate the ssDNA adapter to the 3′-OH end of a bisulfite-converted ssDNA molecule, wherein the 5′-end of the adapter is phosphorylated and the bisulfite-converted ssDNA has been dephosphorylated (i.e., the 3′ end has a hydroxyl group). In another embodiment, the ssDNA ligation reaction uses Thermostable 5′ AppDNA/RNA ligase (available from New England BioLabs (Ipswich, Mass.)) to ligate the ssDNA adapter to the 3′-OH end of a bisulfite-converted ssDNA molecule. In this example, the first UMI adapter is adenylated at the 5′-end and blocked at the 3′-end. In another embodiment, the ssDNA ligation reaction uses a T4 RNA ligase (available from New England BioLabs) to ligate the ssDNA adapter to the 3′-OH end of a bisulfite-converted ssDNA molecule.

In a second step, a second strand DNA is synthesized in an extension reaction. For example, an extension primer, that hybridizes to a primer sequence included in the ssDNA adapter, is used in a primer extension reaction to form a double-stranded bisulfite-converted DNA molecule. Optionally, in some embodiments, the extension reaction uses an enzyme that is able to read through uracil residues in the bisulfite-converted template strand.

Optionally, in a third step, a dsDNA adapter is added to the double-stranded bisulfite-converted DNA molecule. Then, the double-stranded bisulfite-converted DNA can be amplified to add sequencing adapters. For example, PCR amplification using a forward primer that includes a P5 sequence and a reverse primer that includes a P7 sequence is used to add P5 and P7 sequences to the bisulfite-converted DNA. Optionally, during library preparation, unique molecular identifiers (UMI) can be added to the nucleic acid molecules (e.g., DNA molecules) through adapter ligation. The UMIs are short nucleic acid sequences (e.g., 4-10 base pairs) that are added to ends of DNA fragments during adapter ligation. In some embodiments, UMIs are degenerate base pairs that serve as a unique tag that can be used to identify sequence reads originating from a specific DNA fragment. During PCR amplification following adapter ligation, the UMIs are replicated along with the attached DNA fragment, which provides a way to identify sequence reads that came from the same original fragment in downstream analysis.

In an optional step 325, the nucleic acids (e.g., fragments) can be hybridized. Hybridization probes (also referred to herein as “probes”) may be used to target, and pull down, nucleic acid fragments informative for disease states. For a given workflow, the probes can be designed to anneal (or hybridize) to a target (complementary) strand of DNA or RNA. The target strand can be the “positive” strand (e.g., the strand transcribed into mRNA, and subsequently translated into a protein) or the complementary “negative” strand. The probes can range in length from 10s, 100s, or 1000s of base pairs. Moreover, the probes can cover overlapping portions of a target region.

In an optional step 330, the hybridized nucleic acid fragments are captured and can be enriched, e.g., amplified using PCR. In some embodiments, targeted DNA sequences can be enriched from the library. This is used, for example, where a targeted panel assay is being performed on the samples. For example, the target sequences can be enriched to obtain enriched sequences that can be subsequently sequenced. In general, any known method in the art can be used to isolate, and enrich for, probe-hybridized target nucleic acids. For example, as is well known in the art, a biotin moiety can be added to the 5′-end of the probes (i.e., biotinylated) to facilitate isolation of target nucleic acids hybridized to probes using a streptavidin-coated surface (e.g., streptavidin-coated beads).

In step 335, sequence reads are generated from the nucleic acid sample, e.g., enriched sequences. Sequencing data can be acquired from the enriched DNA sequences by known means in the art. For example, the method can include next generation sequencing (NGS) techniques including synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), or paired-end sequencing. In some embodiments, massively parallel sequencing is performed using sequencing-by-synthesis with reversible dye terminators.

In step 340, the sequence processor 210 can generate methylation information using the sequence reads. A methylation state vector can then be generated using the methylation information determined from the sequence reads.

FIG. 3B is an illustration of a process 360 to obtain a methylation information and methylation state vectors, according to an embodiment. The process 360 may be a part of the process 300 described in FIG. 3A. As an example, the analytics system receives a cfDNA molecule 312 that, in this example, contains three CpG sites. As shown, the first and third CpG sites of the cfDNA molecule 312 are methylated 314. During the treatment step 315, the cfDNA molecule 312 is converted to generate a converted cfDNA molecule 322. During the treatment 315, the second CpG site which was unmethylated has its cytosine converted to uracil. However, the first and third CpG sites were not converted.

After conversion, a sequencing library 330 is prepared and sequenced generating a sequence read 342. The analytics system aligns (not shown) the sequence read 342 to a reference genome 344. The reference genome 344 provides the context as to what position in a human genome the fragment cfDNA originates from. In this simplified example, the analytics system aligns the sequence read 342 such that the three CpG sites correlate to CpG sites 23, 24, and 25 (arbitrary reference identifiers used for convenience of description). The analytics system thus generates information both on methylation status of all CpG sites on the cfDNA molecule 312 and the position in the human genome that the CpG sites map to. As shown, the CpG sites on sequence read 342 which were methylated are read as cytosines. In this example, the cytosines appear in the sequence read 342 only in the first and third CpG site which allows one to infer that the first and third CpG sites in the original cfDNA molecule were methylated. Whereas, the second CpG site is read as a thymine (U is converted to T during the sequencing process), and thus, one can infer that the second CpG site was unmethylated in the original cfDNA molecule. With these two pieces of information, the methylation status and location, the analytics system generates 200 a methylation state vector 352 for the fragment cfDNA 312. In this example, the resulting methylation state vector 352 is <M23, U24, M25>, wherein M corresponds to a methylated CpG site, U corresponds to an unmethylated CpG site, and the subscript number corresponds to a position of each CpG site in the reference genome.

In some embodiments, one or more methylation state vectors 352 each has a plurality of contiguous methylation sites. The number of methylation sites can be fixed for some of the methylation state vectors 352. For example, the number may be five in some embodiments, but other numbers may also be used. Contiguous methylation sites may refer to sites that are in the same genetic locus. For example, those sites can be CpG sites that are consecutive. In some embodiments, contiguous sites, or consecutive sites, do not necessarily mean two sites are the immediate neighbor on the DNA sequence level. Instead, in some embodiments, the next contiguous site may refer to the next methylation state that is found in the sequence. Two contiguous sites can be ten or hundred base apart.

### III. IDENTIFYING ANOMALOUS FRAGMENTS

In some embodiments, the analytics system determines anomalous fragments for a sample using the sample's methylation state vectors. For example, for each nucleic acid molecule or fragment in a sample, the analytics system determines whether the nucleic acid molecule or fragment is an anomalously methylated molecule or fragment (via analysis of sequence reads derived therefrom), relative to an expected methylation state vector from a non-cancer sample using the methylation state vector corresponding to the nucleic acid molecule. In one embodiment, the analytics system calculates a p-value score for each methylation state vector describing a probability of observing that methylation state vector or other methylation state vectors even less probable in the non-cancer control group (as described, for example, in U.S. Pat. Appl. Pub. No. 2019/0287652, which is incorporated herein by reference). The process for calculating a p-value score will also be discussed below in Section III.A. P-Value Filtering. The analytics system may determine, and optionally filter out, sequence reads of nucleic acid molecules or fragments with a methylation state vector having below a threshold p-value score as anomalous fragments. In another embodiment, the analytics system further labels fragments with at least some number of CpG sites that have over some threshold percentage of methylation or unmethylation as hypermethylated and hypomethylated fragments, respectively. A hypermethylated fragment or a hypomethylated fragment may also be referred to as an unusual fragment with extreme methylation (UFXM). In other embodiments, the analytics system may implement various other probabilistic models for determining anomalous molecules or fragments. Examples of other probabilistic models include a mixture model, a deep probabilistic model, etc. In some embodiments, the analytics system may use any combination of the processes described below for identifying anomalous fragments. With the identified anomalous fragments, the analytics system may filter the set of methylation state vectors for a sample for use in other processes, e.g., for use in training and deploying a cancer classifier.

### III.A. P-Value Filtering

In one embodiment, the analytics system calculates a p-value score for each methylation state vector compared to methylation state vectors from fragments in a non-cancer control group. The non-cancer control group may comprise healthy samples (e.g., from healthy individuals without any disease diagnosis) or other non-cancer samples (e.g., from individuals without cancer diagnoses but may include other diagnoses). The p-value score describes a probability of observing a nucleic acid molecule having the methylation status matching that methylation state vector in the non-cancer control group. In order to determine a DNA fragment to be anomalously methylated, the analytics system uses a non-cancer control group with a majority of fragments that are normally methylated. When conducting this probabilistic analysis for determining anomalous fragments, the determination holds weight in comparison with the group of control subjects that make up the non-cancer control group. To ensure robustness in the non-cancer control group, the analytics system may select some threshold number of healthy individuals to source samples including DNA fragments. FIG. 4A below describes the method of generating a data structure for a non-cancer control group with which the analytics system can calculate p-value scores. FIG. 4B describes the method of calculating a p-value score with the generated data structure.

FIG. 4A is a flowchart describing a process 400 of generating a data structure for a non-cancer control group, according to an embodiment. To create a non-cancer control group data structure, the analytics system receives a plurality of DNA fragments (e.g., cfDNA) from a plurality of healthy individuals and/or non-cancer individuals. A methylation state vector is identified for each fragment, for example via the process 300 and/or the process 360.

With each fragment's methylation state vector, the analytics system subdivides 405 the methylation state vector into strings of CpG sites. In one embodiment, the analytics system subdivides 405 the methylation state vector such that the resulting strings are all less than a given length. For example, a methylation state vector of length 11 may be subdivided into strings of length less than or equal to 3 would result in 9 strings of length 3, 10 strings of length 2, and 11 strings of length 1. In another example, a methylation state vector of length 7 being subdivided into strings of length less than or equal to 4 would result in 4 strings of length 4, 5 strings of length 3, 6 strings of length 2, and 7 strings of length 1. If a methylation state vector is shorter than or the same length as the specified string length, then the methylation state vector may be converted into a single string containing all of the CpG sites of the vector.

The analytics system 200 tallies 410 the strings by counting, for each possible CpG site and possibility of methylation states in the vector, the number of strings present in the control group having the specified CpG site as the first CpG site in the string and having that possibility of methylation states. For example, at a given CpG site and considering string lengths of 3, there are 2\hat{ }3 or 8 possible string configurations. At that given CpG site, for each of the 8 possible string configurations, the analytics system tallies 410 how many occurrences of each methylation state vector possibility come up in the control group. Continuing this example, this may involve tallying the following quantities: <Mₓ, Mₓ₊₁, Mₓ₊₂<Mₓ, Mₓ₊₁, Uₓ₊₂>, . . . , <Uₓ, Uₓ₊₁, Uₓ₊₂> for each starting CpG site x in the reference genome. The analytics system creates 415 the data structure storing the tallied counts for each starting CpG site and string possibility.

There are several benefits to setting an upper limit on string length. First, depending on the maximum length for a string, the size of the data structure created by the analytics system can dramatically increase in size. For instance, maximum string length of 4 means that every CpG site has at the very least 2\hat{ }4 numbers to tally for strings of length 4. Increasing the maximum string length to 5 means that every CpG site has an additional 2\hat{ } 4 or 16 numbers to tally, doubling the numbers to tally (and computer memory required) compared to the prior string length. Reducing string size helps keep the data structure creation and performance (e.g., use for later accessing as described below), in terms of computational and storage, reasonable. Second, a statistical consideration to limiting the maximum string length is to avoid overfitting downstream models that use the string counts. If long strings of CpG sites do not, biologically, have a strong effect on the outcome (e.g., predictions of anomalousness that predictive of the presence of cancer), calculating probabilities based on large strings of CpG sites can be problematic as it requires a significant amount of data that may not be available, and thus would be too sparse for a model to perform appropriately. For example, calculating a probability of anomalousness/cancer conditioned on the prior 100 CpG sites would require counts of strings in the data structure of length 100, ideally some matching exactly the prior 100 methylation states. If only sparse counts of strings of length 100 are available, there will be insufficient data to determine whether a given string of length of 100 in a test sample is anomalous or not.

FIG. 4B is a flowchart describing a process 420 for identifying anomalously methylated fragments from an individual, according to an embodiment. In process 420, the analytics system generates methylation state vectors from cfDNA fragments of the subject, e.g., by the process 300 and/or the process 360. The analytics system handles each methylation state vector as follows.

For a given methylation state vector, the analytics system enumerates 430 all possibilities of methylation state vectors having the same starting CpG site and same length (i.e., set of CpG sites) in the methylation state vector. As each methylation state is generally either methylated or unmethylated there are effectively two possible states at each CpG site, and thus the count of distinct possibilities of methylation state vectors depends on a power of 2, such that a methylation state vector of length n would be associated with 2ⁿ possibilities of methylation state vectors. With methylation state vectors inclusive of indeterminate states for one or more CpG sites, the analytics system may enumerate 430 possibilities of methylation state vectors considering only CpG sites that have observed states.

The analytics system calculates 440 the probability of observing each possibility of methylation state vector for the identified starting CpG site and methylation state vector length by accessing the non-cancer control group data structure. In one embodiment, calculating the probability of observing a given possibility uses a Markov chain probability to model the joint probability calculation. In other embodiments, calculation methods other than Markov chain probabilities are used to determine the probability of observing each possibility of methylation state vector.

The analytics system calculates 450 a p-value score for the methylation state vector using the calculated probabilities for each possibility. In one embodiment, this includes identifying the calculated probability corresponding to the possibility that matches the methylation state vector in question. Specifically, this is the possibility having the same set of CpG sites, or similarly the same starting CpG site and length as the methylation state vector. The analytics system sums the calculated probabilities of any possibilities having probabilities less than or equal to the identified probability to generate the p-value score.

This p-value represents the probability of observing the methylation state vector of the fragment or other methylation state vectors even less probable in the non-cancer control group. A low p-value score, thereby, generally corresponds to a methylation state vector which is rare in a healthy or non-cancer individual, and which causes the fragment to be labeled anomalously methylated, relative to the non-cancer control group. A high p-value score generally relates to a methylation state vector is expected to be present, in a relative sense, in a healthy or non-cancer individual. If the non-cancer control group is a non-cancerous group, for example, a low p-value indicates that the fragment is anomalous methylated relative to the non-cancer group, and therefore possibly indicative of the presence of cancer in the test subject.

As above, the analytics system calculates p-value scores for each of a plurality of methylation state vectors, each representing a cfDNA fragment in the test sample. To identify which of the fragments are anomalously methylated, the analytics system may filter 460 the set of methylation state vectors based on their p-value scores. In one embodiment, filtering is performed by comparing the p-values scores against a threshold and keeping only those fragments below the threshold. This threshold p-value score could be on the order of 0.1, 0.01, 0.001, 0.0001, or similar.

According to example results from the process, the analytics system yields a median (range) of 2,800 (1,500-12,000) fragments with anomalous methylation patterns for participants without cancer in training, and a median (range) of 3,000 (1,200-220,000) fragments with anomalous methylation patterns for participants with cancer in training. These filtered sets of fragments with anomalous methylation patterns may be used for the downstream analyses as described below.

In one embodiment, the analytics system uses 455 a sliding window to determine possibilities of methylation state vectors and calculate p-values. Rather than enumerating possibilities and calculating p-values for entire methylation state vectors, the analytics system enumerates possibilities and calculates p-values for only a window of sequential CpG sites, where the window is shorter in length (of CpG sites) than at least some fragments (otherwise, the window would serve no purpose). The window length may be static, user determined, dynamic, or otherwise selected.

In calculating p-values for a methylation state vector larger than the window, the window identifies the sequential set of CpG sites from the vector within the window starting from the first CpG site in the vector. The analytic system calculates a p-value score for the window including the first CpG site. The analytics system then “slides” the window to the second CpG site in the vector, and calculates another p-value score for the second window. Thus, for a window size l and methylation vector length m, each methylation state vector will generate m−l+1 p-value scores. After completing the p-value calculations for each portion of the vector, the lowest p-value score from all sliding windows is taken as the overall p-value score for the methylation state vector. In another embodiment, the analytics system aggregates the p-value scores for the methylation state vectors to generate an overall p-value score.

Using the sliding window helps to reduce the number of enumerated possibilities of methylation state vectors and their corresponding probability calculations that would otherwise need to be performed. To give a realistic example, it is possible for fragments to have upwards of 54 CpG sites. Instead of computing probabilities for 2\hat{ }54 (˜1.8×10\hat{ }16) possibilities to generate a single p-score, the analytics system can instead use a window of size 5 (for example) which results in 50 p-value calculations for each of the 50 windows of the methylation state vector for that fragment. Each of the 50 calculations enumerates 2\hat{ }5 (32) possibilities of methylation state vectors, which total results in 50×2\hat{ }5 (1.6×10\hat{ }3) probability calculations. This results in a vast reduction of calculations to be performed, with no meaningful hit to the accurate identification of anomalous fragments.

In embodiments with indeterminate states, the analytics system may calculate a p-value score summing out CpG sites with indeterminates states in a fragment's methylation state vector. The analytics system identifies all possibilities that have consensus with the all methylation states of the methylation state vector excluding the indeterminate states. The analytics system may assign the probability to the methylation state vector as a sum of the probabilities of the identified possibilities. As an example, the analytics system calculates a probability of a methylation state vector of <M₁, I₂, U₃> as a sum of the probabilities for the possibilities of methylation state vectors of <M₁, M₂, U₃> and <M₁, U₂, U₃> since methylation states for CpG sites 1 and 3 are observed and in consensus with the fragment's methylation states at CpG sites 1 and 3. This method of summing out CpG sites with indeterminate states uses calculations of probabilities of possibilities up to 2\hat{ }i, wherein i denotes the number of indeterminate states in the methylation state vector. In additional embodiments, a dynamic programming algorithm may be implemented to calculate the probability of a methylation state vector with one or more indeterminate states. Advantageously, the dynamic programming algorithm operates in linear computational time.

In one embodiment, the computational burden of calculating probabilities and/or p-value scores may be further reduced by caching at least some calculations. For example, the analytic system may cache in transitory or persistent memory calculations of probabilities for possibilities of methylation state vectors (or windows thereof). If other fragments have the same CpG sites, caching the possibility probabilities allows for efficient calculation of p-score values without needing to re-calculate the underlying possibility probabilities. Equivalently, the analytics system may calculate p-value scores for each of the possibilities of methylation state vectors associated with a set of CpG sites from vector (or window thereof). The analytics system may cache the p-value scores for use in determining the p-value scores of other fragments including the same CpG sites. Generally, the p-value scores of possibilities of methylation state vectors having the same CpG sites may be used to determine the p-value score of a different one of the possibilities from the same set of CpG sites.

### III.B Hypermethylated Fragments and Hypomethylated Fragments

In some embodiments, the analytics system determines anomalous fragments as fragments with over a threshold number of CpG sites and either with over a threshold percentage of the CpG sites methylated or with over a threshold percentage of CpG sites unmethylated; the analytics system identifies such fragments as hypermethylated fragments or hypomethylated fragments. Example thresholds for length of fragments (or CpG sites) include more than 3, 4, 5, 6, 7, 8, 9, 10, etc. Example percentage thresholds of methylation or unmethylation include more than 80%, 85%, 90%, or 95%, or any other percentage within the range of 50%-100%.

### III.C. Blocks of Reference Genome

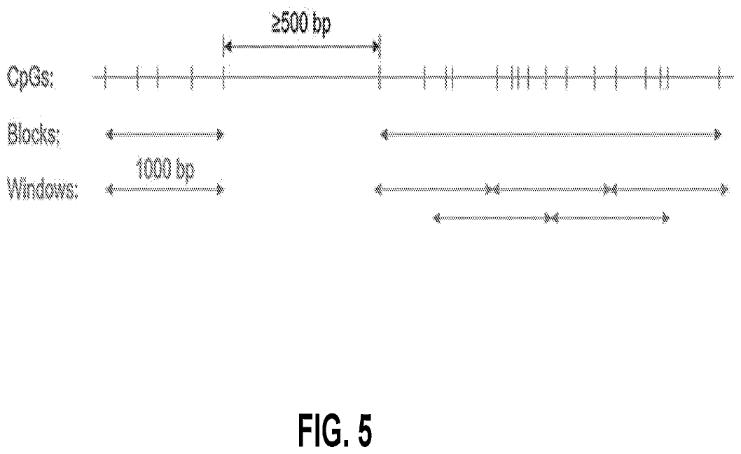

FIG. 5 is an illustration of blocks of a reference genome, according to an embodiment. The sequence processor 210 can partition a reference genome (or a subset of the reference genome) in one or more stages, e.g., for use cases involving a targeted methylation assay. For instance, the sequence processor 210 separates the reference genome into blocks of methylation sites (e.g., CpG sites). Each block is defined when there is a separation between two adjacent CpG sites that exceeds a threshold, e.g., greater than 200 base pairs (bp), 300 bp, 400 bp, 500 bp, 600 bp, 700 bp, 800 bp, 900 bp, or 1,000 bp, among other values. Thus, blocks can vary in size of base pairs. For each block, the sequence processor 210 can subdivide the block into windows of a certain length, e.g., 500 bp, 600 bp, 700 bp, 800 bp, 900 bp, 1,000 bp, 1,100 bp, 1,200 bp, 1,300 bp, 1,400 bp, or 1,500 bp, among other values. In other embodiments, the windows can be from 200 bp to 10 kilobase pairs (kbp), from 500 bp to 2 kbp, or about 1 kbp in length. Windows (e.g., that are adjacent) can overlap by a number of base pairs or a percentage of the length, e.g., 10%, 20%, 30%, 40%, 50%, or 60%, among other values. Windows can be separated between two adjacent CpG sites that exceeds a threshold, e.g., greater than 200 base pairs (bp), 300 bp, 400 bp, 500 bp, 600 bp, 700 bp, 800 bp, 900 bp, or 1,000 bp, among other values.

The sequence processor 210 can analyze sequence reads derived from DNA fragments using a windowing process. In particular, the sequence processor 210 scans through the blocks window-by-window and reads fragments within each window. The fragments can originate from tissue and/or high-signal cfDNA. High-signal cfDNA samples can be determined by a binary classification model, by cancer stage, or by another metric. By partitioning the reference genome (e.g., using blocks and windows), the sequence processor 210 can facilitate computational parallelization. Moreover, the sequence processor 210 can reduce computational resources to process a reference genome by targeting the sections of base pairs that include CpG sites, while skipping other sections that do not include CpG sites.

### IV. CANCER CLASSIFICATION MODEL

FIG. 6 is a flowchart of a method 600 for identifying a plurality of features for generating a classifier to predict a disease state (e.g., presence or absence of a disease, type of disease, and/or a disease tissue of origin), according to various embodiments. In some embodiments, the analytics system 200 performs the method 600 to process sequence reads of fragments from nucleic acid samples. The method 600 includes, but is not limited to, the following steps: generating 610 sequence reads; training 620 variant models associated with each of a plurality of different disease states (e.g., different cancer types); applying 630 the variant models to determine a value based on a probability that a sequence read originated from a sample associated with each of the plurality of disease states associated with each variant model; identifying 640 features by determining a count of sequence reads having a value exceeding a threshold; training 650 a classifier using the features, and optionally applying 660 the classifier to predicting disease state and/or a tissue of origin, associated with a disease state. Each of which are described with respect to the components of the analytics system 200.

The analytics system 200 generates 610 a first set of sequence reads from a plurality of samples each having a known or suspected disease state, such as a presence or absence of a disease, a type of disease, and/or a disease tissue of origin. For example, in some embodiments, the plurality of samples can include any number of cancer samples from individuals known to have cancer and/or non-cancer samples from individuals without cancer. Additionally, the samples can include any of cell free nucleic acid samples (e.g., cfDNA), solid tumor samples, and/or other types of samples.

The analytics system 200 trains 620 the variant models, each associated with a different disease state. The analytic system 200 separate out cohorts of known disease states to train the variant models. To train the variant model, the analytics system 200 may build a distribution based on counts of methylation variants across genomic regions in the cohort for a given disease state. The trained variant model may be configured to input a methylation sequence read and output a probability that the methyl ation sequence read originated from a disease state. In one or more embodiments, there can be two disease states: non-cancer and cancer, i.e., resulting in two variant models. In other embodiments, there can be more disease states: non-cancer, first cancer type, second type, etc. The analytics system 200 may train each variant model based on methylation sequence reads of each cohort.

The analytics system 200 identifies 640 features by determining a count of methylation sequence reads having a value exceeding a threshold value. For example, the variant model for cancer inputs a methylation sequence read and outputs a probability that the methylation sequence read originated from a cancer tissue or cell. The analytics system 200 may set a threshold probability before counting the methylation sequence read for the feature. In some example implementations, the threshold value is selected as 20%, 25%, 30%, 35%, 40%, 45%, 50%, 55%, 60%, 65%, 70%, 75%, 80%, 85%, 90%, 95%.

The analytics system 200 trains 650 a cancer classifier using the features. The analytics system 200 may train the cancer classifier using a cancer cohort of cancer training samples and a non-cancer cohort of non-cancer training samples. The analytics system 200 may featurize each training sample resulting in a feature vector according to step 640. The analytic system 200 then trains the cancer classifier to determine a cancer prediction based on an input feature vector. The analytic system 200 may train the cancer classifier as a machine-learning model. In one or more embodiments, the cancer training samples may be of a known cancer type, such that the analytics system 200 may train the cancer classifier to further predict a tissue of origin in the cancer prediction.

The analytics system 200 may utilize the trained cancer classifier to predict 660 a disease state and/or a tissue of origin, associated with a disease state, of a test sample by applying the cancer classifier. The analytics system 200 may featurize the test sample, resulting in a test feature vector, according to the step 640 using the variant models. The analytics system 200 may apply the cancer classifier to the test feature vector to determine a cancer prediction, i.e., inputting the test feature vector into the cancer classifier, which outputs the cancer prediction. The cancer prediction may be a binary prediction: positive disease state, or negative disease state. The cancer prediction may, in addition or alternatively, be a multiclass prediction: no disease state, first disease state, second disease state, etc.

In some embodiments, the analytics system 200 may train the cancer classifier according to the type of samples used. For example, the analytics system 200 may train the cancer classifier in one manner according to blood samples, and in another manner according to urine samples. Training differently according to the type of samples used ensures that variation in levels of cell-free DNA present in different types of samples do not bias the cancer prediction. For example, a first type of samples may have higher levels of cfDNA compared to a second type of samples. A cancer classifier trained on the first type of samples, then applied to a sample of the second type may skew the cancer prediction.

In some embodiments, the analytics system 200 may train the cancer classifier to incorporate other types of features, in addition to methylation features described above. Other example features include fragment length, endpoint information to enhance understanding of fragments being tumor derived or not tumor derived, protein readouts or mutations, other genetic material including RNA, covariates, or some combination thereof. The other features may be incorporated as inputs of the cancer classifier, along with the methylation features as inputs. In other embodiments, the other features may be used pre-classification and/or or post-classification. In pre-classification, the other features may be used to classify samples into one or more categories, e.g., based on covariates. For example, the other features may be used to separate samples according to predicted ethnicity, predicted age group, predicted smoking status, etc. In post-classification, the analytics system 200 may train a first classifier based on the methylation features as inputs and a second classifier with inputs consisting of the output from the first classifier and the other features fed into the second classifier. The first classifier and/or the second classifier may be machine-learning models (e.g., gradient boosting).

In some embodiments, the analytics system 200 trains a first classifier (e.g., a cancer classifier) to generate a cancer prediction and a second classifier to predict tumor fraction, methylation variant allele fraction, or some combination thereof. The first classifier may be trained utilizing methylation features, and may further include other features. The first classifier inputs the features and outputs a cancer prediction. The cancer prediction may be binary (presence or absence of cancer, and/or likelihoods thereof) and/or multiclass (presence of one or more cancer types, and/or likelihoods thereof). Based on the cancer prediction, the second classifier inputs the methylation features, and may further include other features, to predict tumor fraction, methylation variant allele fraction, or some combination thereof. In one example embodiment, the cancer classifier ranks cancer types (or tissues of origin) for a particular sample. Based on the ranked cancer types, the analytics system may utilize separately trained classifiers for each ranked cancer type to predict tumor fraction, methylation variant allele fraction, or some combination thereof. Each classifier may be independently trained, with various training parameters and metrics (e.g., sensitivity, specificity, etc.).

### IV.A. Tumor Fraction Model and Bank

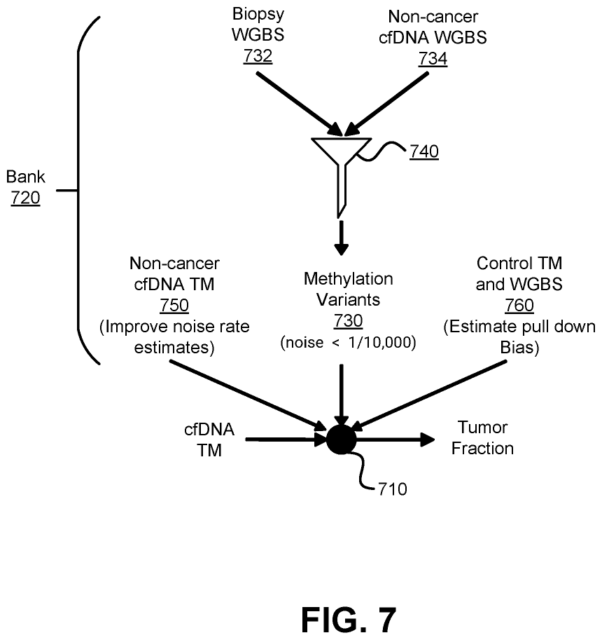

FIG. 7 is a conceptual diagram illustrating how a tumor fraction estimation model 710 may be trained using a bank 720 of reference sequence reads obtained from reference individuals, according to various embodiments. The model 710 may be used to estimate the tumor fraction of a subject using the methylation sequence reads of the subject. The tumor fraction, in this context, may refer to the proportion of fragments (e.g., DNA fragments) in the biological sample of the subject that are tumor derived. In some embodiments, the model 710, or an adjusted version of the model, may also be used to estimate the allele fraction of the subject. In this context, the allele fraction may refer to the proportion of fragments that are tumor derived and contain a variant corresponding to an allele.

In some embodiments, the model 710 used to estimate tumor fraction may be trained based on sequence reads of biological samples of various reference individuals whose disease states are known. A collection of those sequence reads may be referred to as a bank 720 of reference sequence reads. In some embodiments, the reference individuals may include individuals who are diagnosed with one or more cancers and individuals who are healthy (e.g., not diagnosed with cancer). In some embodiments, different biological samples may be obtained to build the bank. For example, some reference biological samples are tissue biopsy samples of individuals while other reference biological samples are cfDNA samples obtained from various types of bodily fluids. In some embodiments, one or more reference individuals may provide more than one type of biological samples while other reference individuals may provide only a single biological sample. For example, a first reference individual may provide a bodily fluid sample and various biopsy samples from different body tissues while a second reference individual may only provide a bodily fluid sample or a single biopsy sample. The bank 720 may include a mix of various individuals with different biological samples provided.

Non-limiting examples of tissue samples include a breast tissue, a thyroid tissue, a lung tissue, a bladder tissue, a cervix tissue, small intestine tissue, a colorectal tissue, an esophagus tissue, a gastric tissue, a tonsil tissue, a liver tissue, an ovary tissue, a fallopian tube tissue, a pancreas tissue, a prostate tissue, a kidney tissue, and a uterus tissue. In some embodiments, the tissue samples may be assigned to one or more groups that comprise similar tissues. For example, a lung adeno and a lung squamous may be separate or may be grouped into a single lung cancer group. Non-limiting examples of bodily fluids include blood, sweat, urine, and saliva. The bank 720 may include sequence reads generated from two, three, four, or all of the different types of tissues. The bank 720 may also include sequence reads of cfDNA samples generated from one or more types of bodily fluids.

In some embodiments, the sequence reads in the bank 720 may be generated using various sequencing methods. For example, for methylation sequencing, whole genome bisulfite sequencing (WGBS) and targeted methylation (TM) assays may be used. Other methods for methylation sequencing, including those disclosed herein and/or any modifications, substitutions, or combinations thereof, can also be used to generate methylation patterns. The sequence reads in the bank 720 may also include control sequence reads for both WGBS and TM assays. Control samples may be sequence reads that are obtained from sequences that are generated randomly using one or more predetermined ratios (e.g., 50:50, 40:60, x60:40, etc.) of methylated sites and unmethylated sites. For example, a control sample may be a 50:50 mix of fully methylated and fully unmethylated sheared genomic DNA. While the bank 720 is discussed primarily with methylation sequencing reads, DNA nucleotide reads may also be included in the bank. For example, in some cases, a methylation sequence read obtained from a biological sample may also have a corresponding DNA nucleotide read obtained from the sample. The corresponding nucleotide read may also be saved in the bank 720.

In some embodiments, the bank 720 includes a collection of methylation variants 730, which can be generated by filtering biopsy WGBS reference samples 732 and non-cancer cfDNA WGBS reference samples 734. In some embodiments, WGBS may take the form of a sequencing process where a nucleic sample undergoes a bisulfite treatment before the converted nucleic acid molecules are evaluated for sequencing information and methylation status on a genome-wide basis. In some embodiments, the whole genome bisulfite sequencing assay looks for variations in methylation patterns in the genome. An example of a WGBS process is described in FIG. 3. CfDNA fragments are obtained from the sample. From the fragments, a location and methylation state for each CpG site is determined based on the alignment of the nucleic acid fragments to a reference genome. A methylation state vector for each fragment may be used to represent information such as the location of the fragment in the reference genome (e.g., as specified by the position of the first CpG site in each fragment, or another similar metric), the number of CpG sites in the fragment, and the methylation state of each CpG site in the fragment. Each methylation state vector, which may include a plurality of potential methylation sites, may be referred to as a methylation variant. In some embodiments, while the term whole genome is used, the WGBS may determine the methylation sequence reads of a part of the genome without analyzing the entirety of the genome. For example, the WGBS may include an analysis of a majority of the genome.

In some embodiments, various biopsy WGBS reference samples 732 and non-cancer cfDNA WGBS reference samples 734 are obtained from different reference individuals. The methylation variants 730 are generated by filtering out methylation variants in the biopsy WGBS reference samples 732 that have a noise rate being higher than a threshold rate. For example, only methylation variants in the biopsy WGBS reference samples 732 that have the noise rate lower than 1/10,000 (or another suitable threshold rate) are kept after passing through the filter 740. The noise rate represents the estimated background noise rate for a particular variant in non-cancer cfDNA data (e.g., using both TM and WGBS non-cancer data). The noise rate may be defined in any suitable statistical manner. In some embodiments, the filtering 740 may be performed by determining a metric for each methylation state vector describing a probability of observing that methylation state vector or other methylation state vectors even less probable in the non-cancer control group. The value of the metric is compared to a threshold value (e.g., 1/10,000).

In some embodiments, the bank 720 also includes targeted methylation (TM) sequence reads 750 of non-cancer cfDNA samples from healthy reference individuals. TM may take the form of an assay that interrogates the methylation status of a discrete set of targets in the genome. In various embodiments, TM sequencing can be performed in different ways. Different enzymatic treatments and combinations with chemical treatment(s) can convert either methylated cytosines or unmethylated cytosines. For example, in some embodiments, the targeted DNA methylation sequencing detects one or more 5-methylcytosine (5mC) and/or 5-hydroxymethylcytosine (5hmC) in the plurality of nucleic acids. As another example, the targeted DNA methylation sequencing may comprise conversion of one or more unmethylated cytosines or one or more methylated cytosines, in the plurality of nucleic acids, to a corresponding one or more uracils. As another example, in some embodiments, the targeted DNA methylation sequencing may comprise conversion of one or more unmethylated cytosines, in the plurality of nucleic acids, to a corresponding one or more uracils, and the DNA methylation sequence reads out the one or more uracils as one or more corresponding thymines. In some embodiments, the targeted DNA methylation sequencing comprises conversion of one or more methylated cytosines, in the plurality of nucleic acids, to one or more corresponding uracils, and the DNA methylation sequence reads out the one or more 5mC or 5hmC as one or more corresponding thymines.

In a TM sequencing process, probes are used to enrich the nucleic acid samples. In some embodiments, probes may be designed such that they bind to sequences after cytosines in methylated CpG sites or un-methylated CpG sites are converted (e.g., in a chemical or enzymatic conversion process). In embodiments in which methylation sequencing is used, sequences of the probes may not be complementary to the corresponding genomic sequence but rather to the sequences of the converted DNA fragments.

The TM sequence reads 750 of non-cancer cfDNA samples can be used to improve noise rate estimates. The TM assay may enrich recurrent methylation variants. The number of methylation variants per block or window of methylation sites in a TM assay can be in an order of magnitude higher than the number of methylation variants in WGBS. As such, the inclusion of TM sequence reads 750 of non-cancer cfDNA samples in the bank 720 can improve the signal-to-noise ratio of target methylation sites, thereby improving the noise rate estimates for the methylation variants 730.

In some embodiments, the bank 720 also includes control TM and WGBS samples 760 to estimate the pull-down biases of various methylation variants. A pull-down bias may be specific to a particular methylation variant. A pull-down bias is typically introduced through the use of probes with particular allelic patterns represented to the exclusion of alternate allelic patterns at a qualifying methylation pattern (QMP) genomic site. In some embodiments, pull-down bias is estimated per QMP genomic site i (biasᵢ), where (biasᵢ) is the pull-down bias at the QMP genomic site i as follows:

$$pc 1 = psuedocount to smooth pull - down bias estimate$$

$$alpha = \frac{{75}^{th} quantile WGBS control ( WGBS count ) abnormal counts}{{75}^{th} quantile TM control ( TM count ) abnormal counts} ,$$

$${bias}_{i} = pull - down bias at QMP genomic site i , and$$

$${bias}_{i} = alpha * ( {x}_{i , TMct} + pc 1 ) / ( {x}_{i , WGBSct} + pc 1 ) .$$

This above-described pull-down bias corrects for pull-down bias in targeted methylation sequencing at a QMP genomic site i using WGBS control data as well as TM control data. In particular, such control data is used to compute alpha. For example, to compute alpha, the abnormal counts at each site in a plurality of QMP genomic sites (understudy) from a WGBS control are obtained (“control (WGBS count) abnormal counts”). As such, there are a plurality of WGBS abnormal counts, each for a different QMP genomic site obtained using the WGBS control. There is no particular requirement on the cancer state of this WGBS control. In other words, the WGBS control can have a particular cancer state or not have a particular cancer state. In some embodiments, the WGBS control is an engineered cell line that has a predetermined known percentage of methylated genomic DNA that is sequenced using WGBS. In some embodiments, the WGBS control is a mixture of 0% methylated and 100% methylated genomic DNA at predetermined compositions (e.g., 50/50 or 40/60 or 30/70 mixture of 0% and 100% methylated genomic DNAs). Further, the abnormal counts at each site in a plurality of QMP genomic sites from a targeted methylation sequencing are obtained (“TM control (TM count) abnormal counts”). In some embodiments, the source of DNA for the TM control is the same as for the WGBS control, the only difference being that, for the TM control, the control DNA is sequenced using targeted sequencing with the pull-down probes used in the TM rather than by WGBS. The quantity alpha in such embodiments may reprsent a slope of a line fitted to a scatterplot of control (WGBS count) abnormal counts/TM control (TM count) abnormal counts. Each respective point in the scatterplot is for a different QMP genomic site j in the plurality of QMP genomic sites under study, where the x coordinate for the respective point is (WGBS count) abnormal counts at genomic site j and they coordinate for respective point is (TM count) abnormal counts at genomic site j. Moreover, as indicated in the equation for alpha, in typical embodiments only data from the 75^{th} quantile of the WGBS control (WGBS count) abnormal counts and only data from the 75^{th} quantile of the TM control (TM count) are used in the scatterplot from which alpha is computed. The quantity alpha, is the slope of a line fitted to the scatterplot data. The use of the 75^{th} quantile is exemplary and that it can be adjusted upwards (e.g., 85^{th} quantile) or downwards (e.g., x65^{th} quantile) in an application-dependent matter. For instance, it can be treated as a hyperparameter that is optimized as part of the optimization of a downstream classifier. Moreover, rather than doing a quantile cut, other methods for removing outliers can be used instead, prior to using the scatterplot to compute alpha.

While the bank 720 is described as having methylation variants 730, the TM sequence reads 750, and the control TM and WGBS samples 760, in various examples the bank 720 may include fewer or additional types of samples. For example, in some embodiments, the bank 720 may include only methylation variants 730. In other embodiments, the bank 720 may include other types of samples that are used to improve various parameters in the model 710, including the noise estimate, the pulldown estimate, the depth estimate, and another suitable parameter.

A biopsy sample typically has over one thousand methylation variants. FIG. 7 is an example plot of the number of methylation variants found per participant, according to some embodiments. Each dot in the plot 700 represents an individual. The median of the methylation variants found per individual is about 2635, according to one experiment. As such, for a potential subject, the number of methylation variants to be found in any biological sample of the subject can be expected to be in the thousand, whether the sample is a biopsy or a cfDNA sample. The large number of methylation variants in a subject allows the methylation variants saved in the bank 720 to be used to build a model 710 that is used to estimate tumor fraction.

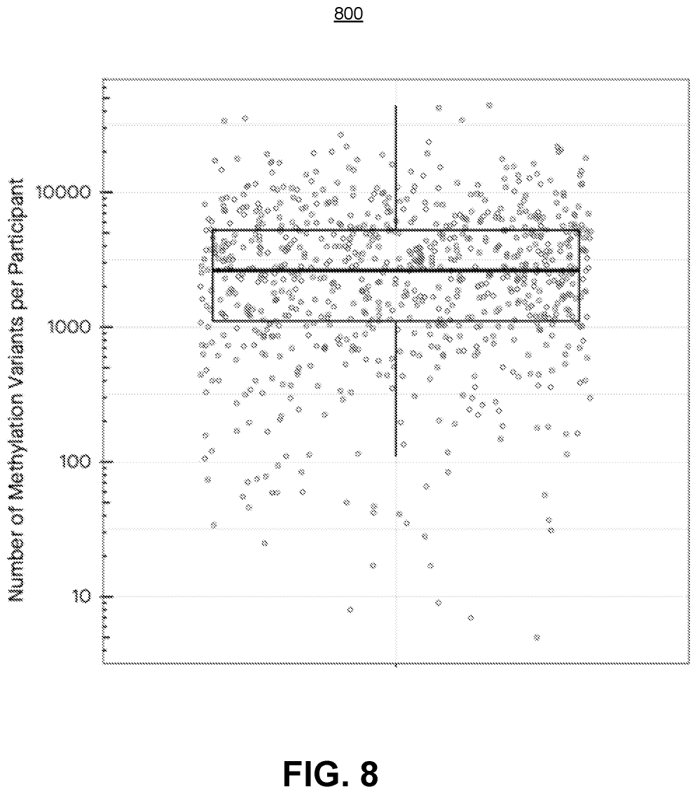

Each methylation variant 730 may be associated with a recurrence rate. A recurrence rate for a given methylation variant may refer to the fraction of the methylation variant presenting in other samples in the bank 720. For example, the recurrence rate of a given methylation variant may be determined based on the number of samples containing one or more supporting fragments that include the methylation variant divided by the total number of samples. In some embodiments, methylation variants are retained using per-participant thresholds for the recurrence rates to be counted. For example, for a particular individual's cancer tissue WGBS sequencing, the analytics system identifies methylation variants that exceed a threshold rate of occurrence within this one sample. Methylation variants that pass this threshold in at least one reference sample are then retained in the plurality of variants. According to various embodiments, the recurrence rate of a methylation variant is expected to be high. FIG. 8 is an example plot of the recurrence rate of various methylation variants found in different participants, according to some embodiments. Each dot in the plot 800 represents a methylation variant. As shown in the plot 800, the vast majority of methylation variants have a recurrence rate of over 0.75. The median recurrence rate is 0.868. As such, the study according to an embodiment shows that methylation variants are recurrent. In other words, a methylation variant that is associated with a disease such as cancer found in one biological sample is expected to be found in other biological samples that are associated with the disease.

In some embodiments, the relatively high recurrence rates of methylation variants and the large number of recurrent methylation variants are used to determine tumor fraction of a subject. The recurrence property is specific to methylation variants and is not observed in other types of variants such as single nucleotide variants, insertions, or deletions. In other words, a methylation variant that is determined by performing sequencing of a biopsy sample of a specific tissue is expected to be found in other tissues of the subject. As such, the presence or absence of a methylation variant may be used to determine the cancer status of the subject. The methylation variant need not be determined from the tissue that is the origin of cancer since the methylation variant is expected to also be observed in other tissues. The methylation variant is also expected to be observed from a cfDNA sample. Based on the high recurrence rates for the majority of methylation variants found in a biological sample, the methylation variants in a cfDNA sample are correlated with the methylation variants in the biopsy sample of a tissue that is suspected to be the origin of cancer. As such, in some embodiments, the tumor fraction of a subject may be estimated using the model 710 with only a cfDNA sample of the subject without a matched biopsy, although in other embodiments a matched biopsy may also be provided. The use of only a cfDNA sample to determine tumor fraction is feasible using methylation variants but can be challenging using other variants such as single nucleotide variants (SNVs) because an SNV often has a low recurrence rate. For example, a cancer-related single nucleotide variant found in a tissue may not necessarily be expected to be found in a cfDNA sample because of a low recurrence rate. Based on the large number of methylation variants and the high recurrence rates expected to be found in individuals, the tumor fraction of a subject may be determined based on the model 710 that factors in the recurrence rates of different methylation variants.

For example, in some embodiments, the model 710 may be trained based on recurrence rates of the methylation variants 730 in the reference sequence reads that are stored in the bank 720. Some methylation variants 730 may be determined to be likely cancer related. In the model 710, the presence or absence of such cancer-related methylation variants in a biological sample is used to determine the tumor fraction of the sample. The relative weight of each methylation variant may be discounted based on the recurrence rate of the methylation variant. For example, the model 710 may be associated with a lower weight for a particular methylation variant if the particular methylation variant has a low recurrence rate because the presence or absence of the methylation variant in the sequenced biological sample does not necessarily mean the same methylation variant is present or absent in another tissue. The training of the model 710 takes account into the recurrence rates of the methylation variants 730. One or more statistical models and distributions may be used to model the correlation between the methylation variants 730 and tumor fraction. The accuracy of the model 710 may be further refined using the non-cancer cfDNA TM sequence reads 750 to improve the noise rate estimates and the control TM and WGBS samples 760 to improve the estimate of the pull-down biases.

The model 710 may be a machine learning model or an algorithmic model that includes a set of rules and one or more statistical models that are used to determine the estimate of the tumor fraction. In various embodiments, the model 710 may take different forms, depending on implementations. For example, the model 710 may include a constant model, a binomial model, a Poisson model, an independent site model, a neural network model, and/or a Markov model. In some embodiments, the model 710 that is trained based on recurrence rates of various methylation variants 730 in the reference sequence reads in the bank 720. In some embodiments, at least part of the model 710 may be expressed as a probabilistic distribution that can be represented as:

$$Prob ( tf \vert data ) ∼ {\prod }_{i = 1}^{n} Poisson ( {x}_{i} ; {λ}_{i} ) \times Prob ( tf )$$

The above equation represents that the conditional probability of the value of tumor fraction (tf) given the data (e.g., cfDNA sample) of a subject is represented as a distribution of the multiply of various methylation-variant-specific Poisson distributions. In the equation above, xᵢ represents the count of a specific methylation variant at site i in the cfDNA sample of the subject; λᵢ represents the Poisson lambda parameter for the site i. The Poisson lambda parameter for site i can be represented as:

λᵢ=[tf×vafᵢ+(1−tf)×noiseᵢ]×depthᵢ

In the above equation, vafᵢ represents the variant allele fraction for site i in the biopsy; noiseᵢ represents the site-specific noise rate in the cfDNA sample; and depthᵢ represents the depth estimate of site i in the cfDNA sample, including those that were not pulled down by the probes. The depth estimate at site i may be affected by the pull-down bias of site i that may be determined based on the control TM and WGBS samples 760 in the bank 720.

In some embodiments, the probability of observing a count of a methylation variant given the tumor fraction value may be expressed as a probabilistic distribution that can be represented as:

Prob(xᵢ|tf)˜[Pᵥ_{ᵢ}Poisson(xᵢ;λᵢ)+(1−Pᵥ_{ᵢ})Poisson(xᵢ; λeᵢ)

In the above equation, Pᵥ_{ᵢ} represents the recurrence rate of a specific methylation variant at site i; eᵢ is the error rate at the site i. As such, λₑ_{ᵢ} is the lambda parameter for a Poisson distribution modeling background noise. λₑ_{ᵢ} may be determined by noiseᵢ×depthᵢ. The recurrence rate may be determined using the sequence reads in the methylation variants 730 in the bank 720. The site-specific noise rate noiseᵢ at the site i may be determined based on the methylation variants 730 and non-cancer cfDNA TM sequence reads 750 in the bank 720. For example, the site-specific noise rate may be determined as the estimated fraction of fragments in the cfDNA sample containing the methylation variant in the absence of cancer.

In some embodiments, the probability of observing a count of a methylation variant given the tumor fraction value may also be assigned or biased by a uniform distribution. For example, the probability of observing a count of a methylation variant given the tumor fraction value may be added with a term α×Uniform(0, dᵢ), where α represents a fraction of likelihood to assign the probability to a uniform distribution and dᵢ represents the fractional counts of fragments overlapping methylation variant i.

In some embodiments, the analytics system may filter methylation variants based on a set of non-cancer samples of interest. In such embodiments, non-cancer tissue data is used to filter out common methylation variants present in non-cancer samples and/or healthy samples. This is advantageous to supplements types of non-cancer samples that may be vulnerable to low levels of signal to noise. For example, the true noise for a particular variant in non-cancer urine samples is 1:10, but low signal could lead to concluding that the variant has noise lower than 1:1000. Knowing that background cfDNA in non-cancer urine is likely coming from bladder, kidney, and/or prostate tissue, the healthy tissue data may be used to further filter the methylation variants to ensure identifying informative methylation variants.

### IV.B Tissue of Origin Fraction Estimation

In some embodiments, the model 710 determines tumor fractions from a plurality of tissue of origins. The model 710 may be a machine-learned model. In some embodiments, the model 710 comprises a plurality of methylation sub-models and a maximum likelihood function that identifies the tumor fraction prediction with maximum likelihood based on the methylation sub-models. The methylation sub-models may be trained based on reference samples (also referred to as training samples) in the bank. The methylation sub-models may be associated with particular methylation variants. For example, a first methylation sub-model may be trained to predict likelihood that a first methylation variant is derived from a particular tissue. The methylation sub-models may further be tailored to subsets of tissues of origin. For example, a methylation model estimates the probability of a fragment coming from a specific cancer tissue having the alternate methylation pattern, and may also estimate the probability of it having the alternate methylation pattern in non-cancer tissue and/or healthy tissue.

In one or more embodiments, the model 710 utilizes a binomial mixture model with a maximum likelihood function to predict the tumor fractions from the plurality of tissue of origins. The binomial mixture model assumes independence between methylation variant sites. In one or more embodiments, the binomial mixture model may be expressed as:

$$P ( VariantModels , {TOO}_{fracs} ) = P ( VariantMode {l}_{0} , {TOO}_{fracs} ) * P ( VariantMode {l}_{1} , {TOO}_{fracs} ) * … * P ( VariantMode {l}_{n} , {TOO}_{fracs} )$$

In the equation above, the probability of observing the counts of the methylation variants over the various methylation variant sites can be independently modeled at each site. The overall probability, shown as P(counts|VariantModels, TOO_{fracs}), is a product of each likelihood of observing the respective count of a methylation variant at each site, shown as P(countₙ|VariantModelₙ, TOO_{fracs}) In other embodiments, other statistical models may be implemented to calculate the likelihood of observing the count of the methylation variant at each site.

In one or more embodiments, the model 710 may identify tumor fractions that maximize the likelihood of the counts given the methylation sub-models for one or more subsets of k number of tissue of origins. The number of tissue of origins k can be any whole number greater than one and less than or equal to the total number of tissue of origins considered. For example, with a total number of tissue of origins M, k can be any one of 2, 3, 4, 5, . . . , M−1, and M. The model 710 may calculate the likelihood of an observed count of a methylation variant at each site using a methylation sub-model generated based on training samples. Types of methylation sub-models that may be implemented include, but are not limited to, a Bernoulli distribution, a binomial distribution, a normal distribution, a Cauchy distribution, a t distribution, a Weibull distribution, etc.

In one or more embodiments, the model 710 calculates the likelihood of observing the count of the methylation variant at a given site using a Poisson distribution. The Poisson distribution is one implementation of a methylation sub-model that expresses the probability of a given number of events occurring in a fixed interval if the events occur at a known constant mean rate and events are independent from one another. A Poisson distribution serves as an approximation of the binomial distribution when the number of events is large, the probability of success of each event is small, but the product of the 2 is not too large or too small. In the context of the methylation variants, the likelihood of observing the count of a methylation variant at a given site can be modeled with a Poisson distribution parameterized by a weighted average of the recurrence rates of the variant across the subset of k tissue of origins.

P(VariantModelᵢ,TOO_{fracs})=Poisson(countᵢ,lambda_TOO_{fracs})

The likelihood of observing the count of a methylation variant at site i, shown as P(countᵢ|VariantModelᵢ, TOO_{fracs}), is modeled as a Poisson distribution with mean parameter lambda_TOO_{fracs} The mean parameter may be calculated as a weighted average based on the predicted tumor fraction across the subset of k tissue of origins and the tissue rate of the methylation variant at that site i for each tissue of origin in the subset. In one embodiment, a modified Poisson distribution outputs densities for any continuous variable (greater than or equal to 0), wherein the density may be represented as a fractional count to each fragment overlapping a methylation variant.

One hypothetical example of the mean parameter calculation follows. At a site, the methylation variant tissue rate for lung tissue of origin samples is 0.4 and the tissue rate for liver is 0.7 and the tissue rate in healthy tissue is 0.1. Suppose an estimated tumor fraction of 0.1 for lung, 0.2 for liver, and 0.7 for healthy tissue. The mean parameter for the estimated tumor fraction and the observed tissue rates is as follows:

$${lambda}_{lung \& liver} = num of fragments * ( {fraction}_{lung} * tissue {rate}_{l u n g} + {fraction}_{l iver} * tissue {rate}_{l iver} + {fraction}_{h ealthy} * tissue {rate}_{h ealthy} )$$

$${lambda}_{lung \& liver} = num of fragments * ( 0.1 * 0.4 + 0.2 * 0.7 + 0.7 * 0.1 )$$

In one or more embodiments, the likelihood of observing the count of a methylation variant at site i further takes into account the likelihood that the methylation variant is turned on or off for a particular sample. The methylation variant may be “on” for a particular tissue when tumor shedding of the methylation variant causes the methylation variant to be present in the cfDNA. The methylation variant may be “off” for a particular tissue when there is no tumor shedding causing absence of the methylation variant from that particular tissue in the cfDNA. One manner of calculating the mean parameter sums over the various possibilities of the tissue of origins in the subset of k tissue of origins being turned on or off. For example with lung and liver, there are four possibilities (or permutations): (1) lung on, liver on; (2) lung on, liver off; (3) lung off, liver on; and (4) lung off, liver off. The likelihood of the methylation being turned on or off for particular tissue of origin at a particular site may be observed over training samples. One manner of calculating the likelihood of an estimated tumor fraction is as follows:

$$P ( VariantMode {l}_{i} , {TOO}_{fracs} ) = {trueSiteRate}_{l u n g} * t r u e SiteRat {e}_{l iver} * Poisson ( {TOO}_{fracs} , {lambda}_{l u n g \& l iver} ) + t r u e S i t e R a t {e}_{l u n g} * ( 1 - t r u e S i t e R a t {e}_{l iver} ) * Poission ( {TOO}_{fracs} , {lambda}_{l u n g} ) + ( 1 - t r u e S i t e R a t {e}_{l u n g} ) * t r u e S i t e R a t {e}_{l iver} * Poisson ( {TOO}_{fracs} , {lambda}_{l iver} ) + ( 1 - t r u e S i t e R a t {e}_{l u n g} ) * ( 1 - t r u e S i t e R a t {e}_{l iver} ) * Poisson ( {TOO}_{fracs} , {lambda}_{h ealthy} )$$

In one or more embodiments, the model 710 performs a basic grid search across all vectors of tumor fractions to identify the estimated tumor fraction that maximizes likelihood. The estimated tumor fraction that maximizes likelihood may be returned by the model 710 as the estimated tumor fraction based on the counts of methylation variants across the sites. The model 710 identifies all combinations of the tissues of origin that are evaluated and performs a basic grid search to identify the tumor fraction for each subset that maximizes the likelihood. The best tumor fractions for all the subsets may then be evaluated to identify the best of the best tumor fraction that maximizes likelihood. In one or more embodiments, the subset size of k is 2. The model 710 identifies all combinations of subsets of two tissues of origin. If, for example, there are 10 total tissue of origins screened for, then there are a total of 45 combinations of two tissues of origin. The model 710 does a first step searching for the best tumor fraction in each subset (e.g., each of the 45 combinations). Then the model 710 compares the best tumor fraction from each subset to identify the best of the best tumor fraction.

In other embodiments, the model 710 may perform a multi-stage search. In a first step, the model 710 predicts a first tissue of origin that the sample is most likely to be derived from. This single tissue of origin prediction may be generated by a multinomial cancer classifier, or other machine learning algorithms capable of predicting a tissue of origin. In some embodiments, the single tissue of origin prediction may be diagnosed by a physician or other healthcare provider. The model 710 may then select combinations of two tissues of origin which include the predicted tissue of origin. The combinations inclusive of the predicted tissue of origin may be grid-searched to estimate a tumor fraction between at least two tissues of origin (including at least the tissue of origin predicted by the classifier). Upon identifying the tumor fraction between two tissues of origin that maximizes likelihood, the model 710 may further increase the subset of tissues of origin evaluated to determine whether a tumor fraction across three tissues of origin results in a larger likelihood than the tumor fraction across two tissues of origin. The model 710 may narrow the searching at each step within some interval of the previous step's estimated tumor fraction.

### IV.C Machine-Learning Implementations

In some embodiment, the model 710 may additionally or alternatively be trained by one or more machine learning techniques. The bank 720 may accumulate a large number of biological samples and the tumor fraction estimates for those samples. The biological samples in the bank 720 may serve as a set of training samples to iteratively train one or more machine learning models that can be used to estimate the tumor fraction of a new cfDNA sample of a subject. Additionally, or alternatively, in some embodiments, one or more parameter values may be determined by one or more machine learning models. For example, the recurrence rate, error rate, pull-down bias, or the depth estimate may be determined using a machine learning model that is trained using the data in the bank 720. For example, each biological sample may be associated with one or more labels, which can be the estimated values of tumor fraction, recurrence rate, error rate, pull-down bias, etc. The biological samples may be used as training samples to iteratively train a machine learning model in a supervised manner.

In various embodiments, a wide variety of machine learning techniques may be used. Examples of which include different forms of unsupervised learning, clustering, embeddings, support vector regression (SVR) model, random forest classifiers, support vector machines (SVMs) such as kernel SVMs, gradient boosting, linear regression, logistic regression, and other forms of regressions. Deep learning techniques such as neural networks, including convolutional neural networks (CNN), recurrent neural networks (RNN), and long short-term memory networks (LSTM), may also be used. Each biological sample may be converted to a feature vector that includes different dimensions. By way of example, the methylation variants in different sites may be expressed as values in different dimensions of the feature vector. The feature vectors of various training samples can be inputted into the machine learning model to iteratively train the machine learning model.

In various embodiments, the training techniques for training a machine learning model may be supervised, semi-supervised, or unsupervised. In supervised training, the machine learning algorithms may be trained with a set of training samples that are labeled. For example, a biological sample in the bank 720 may be labeled with its estimated tumor fraction. The labels for each training sample may be a binary value, a multi-class value, or a continuous variable. In some cases, an unsupervised learning technique may be used. The samples used in training are not labeled. Various unsupervised learning techniques such as clustering may be used. In some cases, the training may be semi-supervised with the training set having a mix of labeled samples and unlabeled samples.

A machine learning model is associated with an objective function, which generates a metric value that describes the objective goal of the training process. For example, the training intends to reduce the error rate of the model in determining the tumor fraction estimate or a parameter value (e.g., recurrence rate, pull-down bias, etc.) in the one or more statistical models mentioned above. In such a case, the objective function may monitor the error rate of the machine learning model. For example, in one machine learning model, the objective function may be the training error rate in predicting the tumor fraction in a training set. In another machine learning model, the objective function may be the training error rate in predicting the recurrence rate of a methylation variant. Such an objective function may be called a loss function. Other forms of objective functions may also be used, particularly for unsupervised learning models whose error rates are not easily determined due to the lack of labels. In various embodiments, the error rate may be measured as cross-entropy loss, L1 loss (e.g., the absolute distance between the predicted value and the actual value), L2 loss (e.g., root mean square distance).

A machine learning model may take various suitable structures. For example, in a neural network, the neural network may receive an input and generate an output. The neural network may include different kinds of layers, such as convolutional layers, pooling layers, recurrent layers, fully connected layers, and custom layers. A convolutional layer convolves the input of the layer with one or more kernels to generate convolved features. Each convolution result may be associated with an activation function. A convolutional layer may be followed by a pooling layer that selects the maximum value (max pooling) or average value (average pooling) from the portion of the input covered by the kernel size. The pooling layer reduces the spatial size of the extracted features. In some embodiments, a pair of convolutional layers and pooling layer 540 may be followed by a recurrent layer that includes one or more feedback loops. The recurrent layer may be gated in the case of an LSTM. The feedback may be used to account for position relationships among methylation variants. The neural network may also include multiple fully connected layers that have nodes connected to each other. The fully connected layers may be used for classification and regression. In some embodiments, one or more custom layers may also be presented for the generation of a specific format of output. The order of layers and the number of layers in the neural network may vary. In some embodiments, a neural network includes one or more convolutional layers but may or may not include any pooling layer or recurrent layer. If a pooling layer is present, not all convolutional layers need to be followed by a pooling layer. A recurrent layer may also be positioned differently at other locations of the neural network. For each convolutional layer, the sizes of kernels (e.g., 3×3, 5×5, 7×7, etc.) and the numbers of kernels allowed to be learned may be different from other convolutional layers.

A machine learning model includes certain layers, nodes, kernels, and/or coefficients. Training of a machine learning model includes iterations of forward propagation and backpropagation. Each layer in a neural network may include one or more nodes, which may be fully or partially connected to other nodes in adjacent layers. In forward propagation, the neural network performs the computation in the forward direction based on outputs of a preceding layer. The operation of a node may be defined by one or more functions. The functions that define the operation of a node may include various computation operations such as convolution of data with one or more kernels, pooling, recurrent loop in RNN, various gates in LSTM, etc. The functions may also include an activation function that adjusts the weight of the output of the node. Nodes in different layers may be associated with different functions.

Each of the functions in the neural network may be associated with different coefficients (e.g. weights and kernel coefficients) that are adjustable during training. In addition, some of the nodes in a neural network each may also be associated with an activation function that decides the weight of the output of the node in forward propagation. Common activation functions may include step functions, linear functions, sigmoid functions, hyperbolic tangent functions (tanh), and rectified linear unit functions (ReLU). After input is provided into the neural network and passes through a neural network in the forward direction, the results may be compared to the training labels or other values in the training set to determine the neural network's performance. The process of prediction may be repeated for other transactions in the training sets to compute the value of the objective function in a particular training round. In turn, the neural network performs backpropagation by using gradient descent such as stochastic gradient descent (SGD) to adjust the coefficients in various functions to improve the value of the objective function.

Multiple iterations of forward propagation and backpropagation may be performed. Training may be completed when the objective function has become sufficiently stable (e.g., the machine learning model has converged) or after a predetermined number of rounds for a particular set of training samples. In some embodiments, a trained machine learning model can be used as the model 710 to predict the tumor fraction using the targeted methylation result of a cfDNA sample as inputs. In some embodiments, a trained machine learning model is used as a sub-model of the model 710 to estimate one or more parameters of the statistical distributions used in the model 710.

### IV.D Exemplary Process

FIG. 10 is a flowchart depicting a process for generating a tumor fraction estimate from a cfDNA sample of a subject, according to some embodiments. The process 1000 may be a computer-implemented process. For example, the flowchart may represent a part of a software algorithm for generating a tumor fraction estimate, according to some embodiments. The software algorithm may be stored as computer instructions that are executable by one or more general processors (e.g., CPUs, GPUs). The instructions, when executed by the processors, cause the processors to perform various steps described in the process 1000. In various embodiments, one or more steps in the process 1000 may be skipped or be changed. The process 1000 may be performed by analytics system 200. In general, the computer-implemented process may be performed using a computer.

In step 1010, a dataset of methylation sequence reads from a cfDNA sample of a subject is received. The dataset of the methylation sequence reads may be generated using the process 300 described in FIG. 3A and/or the process 360 described in FIG. 3B. In some embodiments, the dataset included in estimating the tumor fraction may include methylation sequence reads determined from only a cfDNA sample. As such, the process 1000 may be used to determine the amount of cancer signal in the cfDNA sample of the subject without a reference biopsy. For example, the process 1000 may be used as an early diagnostic technique where the cfDNA sample is obtained from a bodily fluid without an invasive biopsy. In some cases, the subject has not been diagnosed with cancer or the tissue origin of the cancer is still unknown. As such, no biopsy sample is provided for determining the tumor fraction. While the process 1000 can be performed with only a cfDNA sample, in some embodiments, the dataset may also include other sequence reads generated from other biological samples of the subject. For example, a matched biopsy sample may also be presented for providing additional data points in the process 1000.

In some embodiments, the methylation sequence reads in the dataset may be generated based on a targeted methylation (TM) assay. The TM assay may select methylation variant sites that are expected to have high recurrence rates. Using a TM assay, the depth and coverage of the sequencing for the methylation variant sites of interest can also be improved compared to a WGBS. Hence, the noise estimates for the targeted methylation sites can be improved.

In step 1020, the dataset is divided into a plurality of variants. Each variant may be a methylation variant that includes one or more CpG sites. For example, at least one of the variants includes a plurality of contiguous CpG sites. For example, the sequence reads may be divided by a pattern of n (e.g., 5) contiguous CpGs and their states that distinguish DNA derived from one biological source (e.g., cancer) from another (e.g., non-cancer cfDNA). The dataset may be stored in a map of intervals where each key defines a different reference chromosome. The dataset may be referred to as a match tree, which may take the form of a serializable data structure that stores counts of fragments for unique fragment-level methylation patterns. The underlying interval map keys may take the form of intervals defines by the first and last CpG indices of a fragment. The data stored in each interval map entry may be the number of observations of fragments for each unique observed set of methylation states in the interval. In some embodiments, each variant has the same length of CpG sites. For example, each variant includes a length of 5 CpG sites. In other embodiments, the lengths of CpG sites in various variants can be different.

In step 1030, the methylation states of the plurality of variants are determined. In some embodiments, a particular methylation variant may include a plurality of CpG sites (e.g., contiguous CpG sites). The methylation variant may be encoded by a series of binary values. The series corresponds to the CpG sites. For each site, a first binary value (e.g., 1) represents that methylation is observed while a second binary value (e.g., 0) represents that no methylation is observed. The methylation state of a methylation variant may be referred to as a methylation state vector, as described in FIGS. 3A and 3B. For example, a methylation state vector of 11111 represents that all sites in the variant are methylated. Likewise, a methylation state vector of 00000 represents that all sites in the variant are unmethylated. The methylation state of a variant may also be anything between 11111 and 00000. The determination of the methylation states of the variants may also be referred to as a variant calling process. Variants may be called from an input fragment file representing sequenced fragments from a biological sample and a reference non-cancer match tree that is specifically built from WGBS non-cancer cfDNA. The variant calling process may also include fragment filtering. For example, fragments are filtered to remove duplicates, unconverted fragments, and uncalled fragments. Fragments that are too shorted, such as those having a minimal number of CpG sites lower than a typical variant size of 5 CpG sites, may be filtered out. The fragments may also be filtered based on a minimum mapping quality threshold.

In step 1040, the plurality of variants is filtered based on a bank of reference sequence reads to generate a filtered subset of variants. The bank may be the bank 720 described in FIG. 7 and includes reads generated from non-cancer samples and biopsy samples of a plurality of tissues of reference individuals. The filtering that generates a filtered subset of variants may include filtering out one or more methylation variants whose rates of presence in the non-cancer samples exceed a threshold. For example, if a methylation variant is also commonly present in the non-cancer samples in the bank 720, the methylation variant may be associated with a high noise rate and may be filtered out as a result. The filtering of the methylation variants may also be based on other suitable criteria. In some embodiments, methylation variants are filtered to keep those that are not in the top 5% (or another suitable percentage threshold) of raw sample counts. In some embodiments, the filtering may keep only the methylation variants that are in an autosome. In some embodiments, only certain patterns of the methylation state are kept after the filtering. For example, in one embodiment, only methylation variants that are completely methylated or completely unmethylated are kept after the filtering. In some embodiments, the selected methylation variants are those that have at least one matched fragment in the input TM reference data. In some embodiments, the selected methylation variants are those that have a TM estimated noise rate below a certain threshold such as 1/10,000. In some embodiments, the selected methylation variants are those that have a WGBS noise rate below a certain threshold such as 1/10,000.

In step 1050, the counts of the methylation states of variants in the filtered subset are determined. The count of a particular methylation state corresponding to a particular variant may represent a number of variant reads in the dataset that have the particular methylation state. For example, the count at each site may be represented as xᵢ, which stands for the count of a specific methylation variant at site i in the cfDNA sample of the subject. Each methylation variant in the cfDNA sample may be associated with a specific count. The counts for various methylation variants may depend on the sequencing depth. The count may take the form of a fractional count of fragments that contain a specific methylation variant in view of the total number of fragments sequenced. Other parameters such as the depthᵢ, which represents the depth estimate of site i in the cfDNA sample, may also be determined. The depth may also take the form of a fraction count, which represents the fragments that overlap the variant but does not necessarily include the specific methylation state of the variant. After the counts for various methylation variants in the filtered subset are determined, the counts may be stored as a vector that represents the input for a model to determine the tumor fraction estimate.

In step 1060, the counts of methylation states are input into a model that is trained based on recurrence rates of the plurality of variants in the reference sequence reads in the bank. For example, the model may be the model 710 of FIG. 7. The model takes into account the recurrence rates of various methylation variants. A particular recurrence rate of a particular variant may correspond to a rate of observation of the particular variant among the reference sequence reads in the bank. In some embodiments, variant recurrence may be estimated as the number of tissue biopsy samples for a particular label (e.g., breast cancer) that have any evidence of the variant (e.g., the sample having one or more support fragments). The recurrence rate may be estimated as the number of samples in the bank 720 containing one or more supporting fragments that include the methylation variant having the methylation state divided by the total of samples.

The model used to determine the tumor fraction estimate may factor in one or more recurrence rates for various methylation variants. In some embodiments, the model includes a probabilistic model. The probabilistic model includes Poisson distributions for various methylation variants. A particular Poisson distribution for a particular methylation variant may be factored by the recurrence rate of the particular variant. In some embodiments, the model includes a plurality of probabilistic distributions. Each probabilistic distribution may correspond to a particular variant and be parameterized based on a site-specific noise rate of the particular variant and a depth of site of the particular variant. In some embodiments, the model includes a machine learning model. Details of various implementations of the model 710 are described in FIG. 7.

The model may be trained using the data of the bank 720. Some reference individuals in the bank 720 are healthy while other reference individuals in the bank are determined to have cancer or a type of cancer. The cancer or a type of cancer may be a breast cancer, uterine cancer, cervical cancer, ovarian cancer, bladder cancer, urothelial cancer of renal pelvis and ureter, renal cancer other than urothelial, prostate cancer, anorectal cancer, colorectal cancer, squamous cell cancer of esophagus, esophageal cancer other than squamous, gastric cancer, hepatobiliary cancer arising from hepatocytes, hepatobiliary cancer arising from cells other than hepatocytes, pancreatic cancer, human-papillomavirus-associated head and neck cancer, head and neck cancer not associated with human papillomavirus, lung adenocarcinoma, small cell lung cancer, squamous cell lung cancer and lung cancer other than adenocarcinoma or small cell lung cancer, neuroendocrine cancer, melanoma, thyroid cancer, sarcoma, multiple myeloma, lymphoma, or leukemia.

In step 1070, using the model, the tumor fraction estimate of the cfDNA sample is generated. In some embodiments, the tumor fraction estimate may be the output of the model. In various embodiments, the output may take different forms. For example, in some embodiments, the tumor fraction estimate is a distribution of the probability of a fraction of fragments in the cfDNA sample that are tumor derived. In some embodiments, instead of a distribution, the tumor fraction estimate is a score that determines the fraction of fragments that are likely to be tumor derived. In some embodiments, the tumor fraction estimate may also be a binary determination (e.g., the subject is estimated to have cancer or not have cancer) or multi-class determinations (e.g., no cancer, possible to have cancer, probable to have cancer, likely to have cancer, further determination is needed, etc.). In some embodiments, the tumor fraction estimate may be estimated fractions across multiple tissues of origin. The tumor fraction estimate may be used as a signal for estimating the amount of cancer in the cfDNA sample of the subject in the absence of a reference biopsy sample. The tumor fraction estimate may also have other uses such as classification of cancer and determination of tissue origin of cancer.

### V. ADDITIONAL IMPROVEMENTS

### V.A. Example 1—Enriching of Methylation Variants with Targeted Assays

In some embodiments, the methylation variants may be enriched using targeted methylation assays. FIG. 10 includes plots of the number of methylation variants per megabase in a targeted methylation panel and a WGBS panel. The plots show that the TM panel has significantly higher numbers of methylation variants that are in orders of magnitude higher than those numbers in the WGBS panel. The result shows that TM assay can enrich all methylation variants including recurrent methylation variants. This is not necessarily true for other variants such as single nucleotide variants. In some embodiments, a TM assay is used to generate the dataset of methylation sequence reads from the cfDNA sample of a subject in step 910 of process 900.

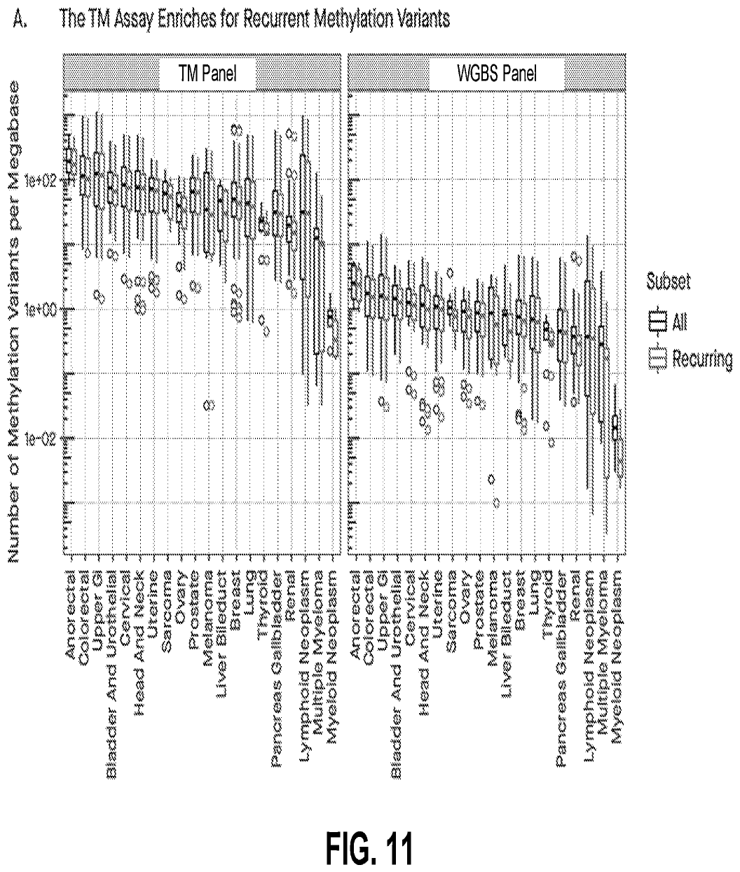

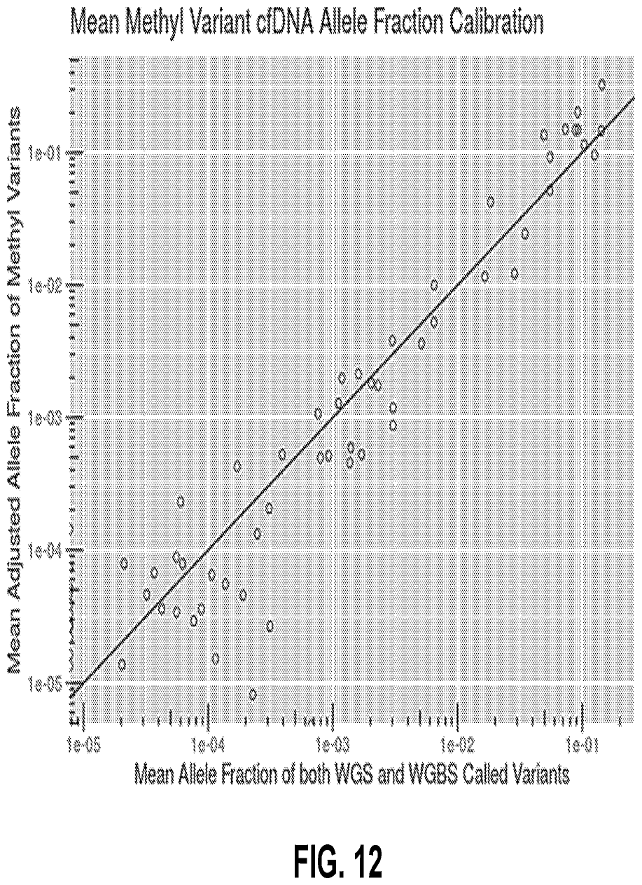

In some embodiments, the tumor fraction estimate may also be used to generate an allele fraction estimate. An allele fraction may refer to the proportion of molecules that are tumor derived and contain a variant. FIG. 11 is a plot that illustrates a mean methylation variant cfDNA allele fraction calibration, according to some embodiments. The mean adjusted allele fraction of methylation variants in the y-axis may be determined based on the allele fraction estimate from tumor fraction multiplied by mean biopsy allele fraction. The mean allele fraction of both WGS (whole genome sequencing) and WGBS called variants in the x-axis may be determined based on the allele fraction estimate from direct counting of fragments containing a confident set of single nucleotide variants (SNVs) called in WGBS biopsy. FIG. 11 shows that the mean adjusted allele fraction from methyl variants is well calibrated against allele fraction from WGS called variants. As such, allele fraction may be directly estimated from tumor fraction.

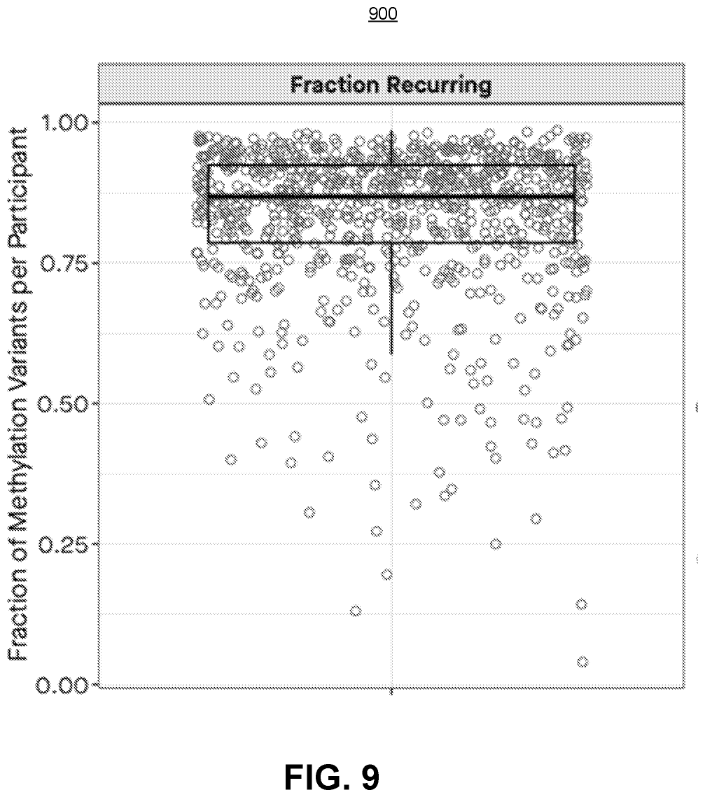

In various embodiments, one or more biological samples described herein, including those samples discussed in FIGS. 6 and 9, may be enriched using a cancer assay panel comprising a plurality of probes or a plurality of probe pairs. A number of targeted cancer assay panels are known in the art, for example, as describe in WO 2019/195268 filed Apr. 2, 2019, PCT/US2019/053509 filed Sep. 27, 2019 and PCT/US2020/015082 filed Jan. 24, 2020 (which are incorporated herein by reference). For example, in some embodiments, the cancer assay panel can be designed to include a plurality of probes (or probe pairs) that can capture fragments that can together provide information relevant to diagnosis of cancer. In some embodiments, a panel includes at least 50, 100, 500, 1,000, 2,000, 2,500, 5,000, 6,000, 7,500, 10,000, 15,000, 20,000, 25,000, or 50,000 pairs of probes. In other embodiments, a panel includes at least 500, 1,000, 2,000, 5,000, 10,000, 12,000, 15,000, 20,000, 30,000, 40,000, 50,000, or 100,000 probes. The plurality of probes together can comprise at least 0.1 million, 0.2 million, 0.4 million, 0.6 million, 0.8 million, 1 million, 2 million, 3 million, 4 million, 5 million, 6 million, 7 million, 8 million, 9 million, or 10 million nucleotides. The probes (or probe pairs) are specifically designed to target one or more genomic regions differentially methylated in cancer and non-cancer samples. The target genomic regions can be selected to maximize classification accuracy, subject to a size budget (which is determined by sequencing budget and desired depth of sequencing).

Samples enriched using a cancer assay panel can be subject to targeted sequencing. Samples enriched using the cancer assay panel can be used to detect the presence or absence of cancer generally and/or provide a cancer classification such as cancer type, stage of cancer such as I, II, III, or IV, or provide the tissue of origin where the cancer is believed to originate. Depending on the purpose, a panel can include probes (or probe pairs) targeting genomic regions differentially methylated between general cancerous (pan-cancer) samples and non-cancerous samples, or only in cancerous samples with a specific cancer type (e.g., lung cancer-specific targets). Specifically, a cancer assay panel is designed based on bisulfite sequencing data generated from the cell-free DNA (cfDNA) or genomic DNA (gDNA) from cancer and/or non-cancer individuals.

In some embodiments, the cancer assay panel designed by methods provided herein comprises at least 1,000 pairs of probes, each pair of which comprises two probes configured to overlap each other by an overlapping sequence comprising a 30-nucleotide fragment. The 30-nucleotide fragment comprises at least five CpG sites, wherein at least 80% of the at least five CpG sites are either CpG or UpG. The 30-nucleotide fragment is configured to bind to one or more genomic regions in cancerous samples, wherein the one or more genomic regions have at least five methylation sites with an abnormal methylation pattern. Another cancer assay panel comprises at least 2,000 probes, each of which is designed as a hybridization probe complimentary to one or more genomic regions. Each of the genomic regions is selected based on the criteria that it comprises (i) at least 30 nucleotides, and (ii) at least five methylation sites, wherein the at least five methylation sites have an abnormal methylation pattern and are either hypomethylated or hypermethylated.

Each of the probes (or probe pairs) is designed to target one or more target genomic regions. The target genomic regions are selected based on several criteria designed to increase selective enriching of relevant cfDNA fragments while decreasing noise and non-specific bindings. For example, a panel can include probes that can selectively bind and enrich cfDNA fragments that are differentially methylated in cancerous samples. In this case, sequencing of the enriched fragments can provide information relevant to diagnosis of cancer. Furthermore, the probes can be designed to target genomic regions that are determined to have an abnormal methylation pattern and/or hypermethylation or hypomethylation patterns to provide additional selectivity and specificity of the detection. For example, genomic regions can be selected when the genomic regions have a methylation pattern with a low p-value according to a Markov model trained on a set of non-cancerous samples, that additionally cover at least 5 CpG's, 90% of which are either methylated or unmethylated. In other embodiments, genomic regions can be selected utilizing mixture models, as described herein.

Each of the probes (or probe pairs) can target genomic regions comprising at least 25 bp, 30 bp, 35 bp, 40 bp, 45 bp, 50 bp, 60 bp, 70 bp, 80 bp, or 90 bp. The genomic regions can be selected by containing less than 20, 15, 10, 8, or 6 methylation sites. The genomic regions can be selected when at least 80, 85, 90, 92, 95, or 98% of the at least five methylation (e.g., CpG) sites are either methylated or unmethylated in non-cancerous or cancerous samples.

Genomic regions may be further filtered to select only those that are likely to be informative based on their methylation patterns, for example, CpG sites that are differentially methylated between cancerous and non-cancerous samples (e.g., abnormally methylated or unmethylated in cancer versus non-cancer). For the selection, calculation can be performed with respect to each CpG site. In some embodiments, a first count is determined that is the number of cancer-containing samples (cancer_count) that include a fragment overlapping that CpG, and a second count is determined that is the number of total samples containing fragments overlapping that CpG (total). Genomic regions can be selected based on criteria positively correlated to the number of cancer-containing samples (cancer_count) that include a fragment overlapping that CpG, and inversely correlated with the number of total samples containing fragments overlapping that CpG (total).

In one embodiment, the number of non-cancerous samples (nnon-cancer) and the number of cancerous samples (ncancer) having a fragment overlapping a CpG site are counted. Then the probability that a sample is cancer is estimated, for example as (ncancer+1)/(ncancer nnon-cancer 2). CpG sites by this metric are ranked and greedily added to a panel until the panel size budget is exhausted.

Depending on whether the assay is intended to be a pan-cancer assay or a single-cancer assay, or depending on what kind of flexibility is desired when picking which CpG sites are contributing to the panel, which samples are used for cancer-count can vary. A panel for diagnosing a specific cancer type (e.g., TOO) can be designed using a similar process. In this embodiment, for each cancer type, and for each CpG site, the information gain is computed to determine whether to include a probe targeting that CpG site. The information gain is computed for samples with a given cancer type compared to all other samples. For example, two random variables, “AF” and “CT”. “AF” is a binary variable that indicates whether there is an abnormal fragment overlapping a particular CpG site in a particular sample (yes or no). “CT” is a binary random variable indicating whether the cancer is of a particular type (e.g., lung cancer or cancer other than lung). One can compute the mutual information with respect to “CT” given “AF.” That is, how many bits of information about the cancer type (lung vs. non-lung in the example) are gained if one knows whether there is an anomalous fragment overlapping a particular CpG site. This can be used to rank CpG's based on how specific they are for a particular cancer type (e.g., TOO). This procedure is repeated for a plurality of cancer types. For example, if a particular region is commonly differentially methylated only in lung cancer (and not other cancer types or non-cancer), CpG's in that region would tend to have high information gains for lung cancer. For each cancer type, CpG sites ranked by this information gain metric, and then greedily added to a panel until the size budget for that cancer type was exhausted.

Further filtration can be performed to select target genomic regions that have off-target genomic regions less than a threshold value. For example, a genomic region is selected only when there are less than 15, 10 or 8 off-target genomic regions. In other cases, filtration is performed to remove genomic regions when the sequence of the target genomic regions appears more than 5, 10, 15, 20, 25, or 30 times in a genome. Further filtration can be performed to select target genomic regions when a sequence, 90%, 95%, 98% or 99% homologous to the target genomic regions, appear less than 15, 10 or 8 times in a genome, or to remove target genomic regions when the sequence, 90%, 95%, 98% or 99% homologous to the target genomic regions, appear more than 5, 10, 15, 20, 25, or 30 times in a genome. This is for excluding repetitive probes that can pull down off-target fragments, which are not desired and can impact assay efficiency.

In some embodiments, fragment-probe overlap of at least 45 bp was demonstrated to be required to achieve a non-negligible amount of pulldown (though this number can be different depending on assay details). Furthermore, it has been suggested that more than a 10% mismatch rate between the probe and fragment sequences in the region of overlap is sufficient to greatly disrupt binding, and thus pulldown efficiency. Therefore, sequences that can align to the probe along at least 45 bp with at least a 90% match rate are candidates for off-target pulldown. Thus, in one embodiment, the number of such regions are scored. The best probes have a score of 1, meaning they match in only one place (the intended target region). Probes with a low score (say, less than 5 or 10) are accepted, but any probes above the score are discarded. Other cutoff values can be used for specific samples.

In various embodiments, the selected target genomic regions can be located in various positions in a genome, including but not limited to exons, introns, intergenic regions, and other parts. In some embodiments, probes targeting non-human genomic regions, such as those targeting viral genomic regions, can be added.

### V.B. Example 2—Analysis of Tumor Fraction in Urine-Derived Cfdna

Urological cancers such as prostate, bladder and kidney cancers, have lower detection sensitivity in the Circulating Cell-free Genome Atlas Study (“CCGA”; Clinical Trial.gov identifier NCT02889978), a prospective, multi-center, case-control, observational study with longitudinal follow-up. This low detection rate may be due to a low tumor fraction of cfDNA in the blood of subjects with urothelial cancers. Analysis of cell-free DNA from urine could increase the sensitivity of detection for urological cancers.

In an exemplary workflow, about 50 mL of urine is collected from a subject. A preservative is then added to the urine sample following collection. Streck Urine Preserve (Streck, Nebr., USA) or an equivalent preservative containing at least 0.5% weight-by-volume (w/v) of a nuclease inhibitor, at least 0.2-4.0% w/v of a preservative agent, and at least 0.01% w/v of a formaldehyde quencher may be used as the preservative. Other preservatives that may be used include the Urine Collection Medium and UAS (Novosantis, Belgium). Alternatively, urine samples may be collected using a urine collection and preservation device (Norgen Biotek Corp., Canada) or an equivalent cup that contains from 10-30% w/v of a nuclease inhibitor such as EDTA, and 0.1-1.0% w/v of a bacteriostatic preservative agent such as sodium azide.

After addition of the preservative, the urine samples are centrifuged at 4000×g for 20 minutes to sediment and remove any cellular debris. The resulting supernatant is then concentrated approximately 15-fold. The concentration of the preserved urine sample may be achieved using diafiltration column, such as a filter unit having a regenerated cellulose membrane with a 3 kDa cut-off. For example, if the resulting supernatant has a volume of 15 mL or lower, the sample may be concentrated by spinning at 4000×g for at least 30 minutes in an Amicon Ultra-15 filter unit (Thermo Fisher Scientific, Massachusetts, US). If working with volumes ranging from 15 mL to 50 mL of supernatant, the sample may be instead concentrated by spinning at 2500×g in a Centricon Plus-70 centrifugal filter unit (Thermo Fisher Scientific, Massachusetts, US), for at least 40 minutes or until the sample has been concentrated to a volume lower than 4.2 mL. The concentration of the urine samples to lower volumes results in samples amenable to automated bead-based extraction methods (e.g., MagMax extraction) and other benchtop accessible techniques.

After concentration of the urine sample, the samples may be immediately used or frozen at −80 C for later use, or for batch processing of samples. The concentrated urine sample may then be subjected to cfDNA extraction and library preparation, which can then be sequenced for methylation analysis and urological cancer detection.

Addition of a preservative no more than 30 minutes after urine sample collection preserved nucleosomes and produced cfDNA fragments of up to 7000 bp in size. In contrast, delaying the addition of the preservative to an hour or more after collection led to loss of nucleosomal peaks at >700 bp, a significant decrease in yield, and a narrower distribution of fragment lengths skewed toward low molecular weight fragments, indicating lysis of cells in urine, along with degradation and fragmentation of the cfDNA. It was also found that by concentrating urine, samples that were frozen for later processing exhibited reduced cryoprecipitate formation.

Cancer-specific methylation signatures, such as those corresponding to samples from subjects with stage I, high-grade non-muscle invasive bladder cancer, were detected in urine cfDNA processed from urine samples.

To evaluate how the detection of cancer-specific methylation status from urine-derived and plasma-derived cfDNA differ, urine samples were collected and processed as described in Example 1. A further study was conducted to evaluate detection of cancer-specific methylation markers in urine-derived cfDNA as compared to plasma tumor fraction estimates. Urine and blood were collected from patients with bladder cancer, renal cancer, and prostate cancer as well as from age- and gender-matched non-cancer patients. To generate biopsy-free estimates in urine cfDNA, a plasma-based workflow was modified at the following steps (illustrated in FIG. 7): (1) an external reference dataset of non-cancer urine cfDNA (N=−200) was used instead of plasma for the non-cancer WGBS and TM data in the workflow; (2) the noise threshold and pseudocount were adjusted to account for the smaller reference datasets; and (3) WGBS data from healthy urological tissue was used to further filter noisy methylation variants.

Sequencing libraries were then prepared from the resulting cfDNA for methylation analysis of a panel of urological cancer methylation markers. Tumor fraction estimation was also performed using the methylation marker panel, and samples with an estimated tumor fraction above a threshold were identified as detected for cancer based on urine-derived cfDNA. The estimated tumor fraction in urine-derived cfDNA sample was then compared to the estimated tumor fraction from a corresponding plasma-derived cfDNA fraction.

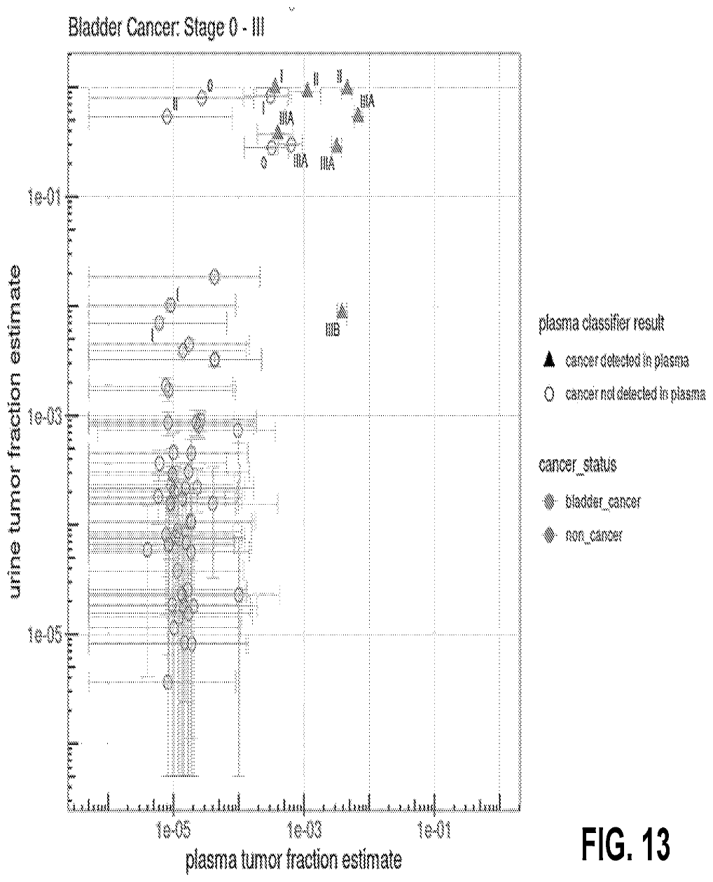

The scatterplots in FIGS. 13-15 show the tumor fraction distribution in each cancer type in matched urine and plasma cfDNA (each point is a single patient). The fill represents whether the multi-cancer classifier (at 99% specificity) detected the plasma cfDNA for that patient. Increased tumor fraction in urine cfDNA relative to plasma was observed in all bladder cancer patients analyzed and in a subset of prostate cancer patients. Plasma tumor fraction estimates were consistent with classifier detection. In kidney cancer patients, while signal in urine was not increased over that of plasma, higher tumor fraction in urine cfDNA relative to non-cancer patients was observed. In a small number of patients, increased signal in plasma relative to urine cfDNA was observed.

The estimated tumor fraction for subjects with bladder cancer of any stage was found to be higher when using urine-derived cfDNA compared to plasma-derived cfDNA (FIG. 13). When evaluating samples from subjects with early-stage, non-muscle invasive bladder cancers, estimates based on urine tumor fraction stratified several bladder cancer samples for which detection in plasma did not detect cancer, along with most of those samples that were also detected in plasma.

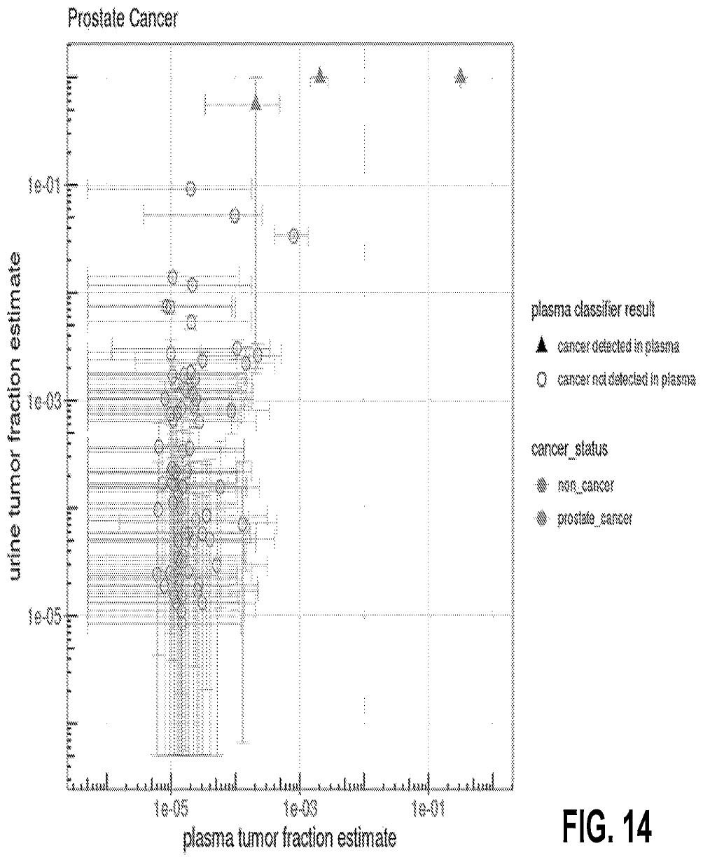

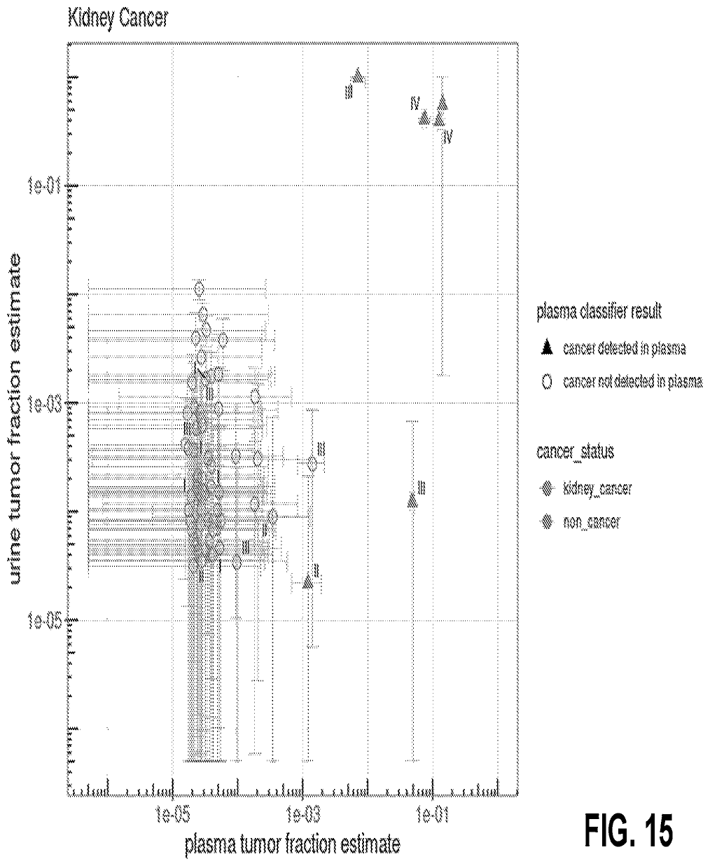

When analyzing a cohort of subjects with stage II or IV prostate cancer, the estimated tumor fraction was also higher in urine-derived cfDNA than in plasma-derived cfDNA (FIG. 14). In particular, five out of the ten samples analyzed had a urine-derived cfDNA estimated tumor fraction above a minimum threshold, while only one of the ten plasma-derived cfDNA estimated tumor fraction was above the threshold. The estimated tumor fraction in samples from subjects with kidney cancer was found to be comparable between urine- and plasma-derived cfDNA (FIG. 15).

In conclusion, determination of urological cancer status based on analysis of methylation markers and tumor fraction was comparable or more accurate when analyzing urine-derived cfDNA than plasma-derived cfDNA. Concentrating urine samples improved the processing and analysis of urine-derived cfDNA for urological cancer detection.

### VI. CANCER APPLICATIONS

In some embodiments, the methods, analytic systems and/or tumor fraction estimate model of the present disclosure can be used to detect the presence (or absence) of cancer, monitor cancer progression or recurrence, monitor therapeutic response or effectiveness, determine a presence or monitor minimum residual disease (MRD), or any combination thereof. In some embodiments, the analytic systems and/or classifier may be used to identify the tissue or origin for a cancer. For instance, the systems and/or classifiers may be used to identify a cancer as of any of the following cancer types: head and neck cancer, liver/bileduct cancer, upper GI cancer, pancreatic/gallbladder cancer; colorectal cancer, ovarian cancer, lung cancer, multiple myeloma, lymphoid neoplasms, melanoma, sarcoma, breast cancer, and uterine cancer. For example, as described herein, a classifier can be used to generate a likelihood or probability score (e.g., from 0 to 100) that a sample feature vector is from a subject with cancer. In some embodiments, the probability score is compared to a threshold probability to determine whether or not the subject has cancer. In other embodiments, the likelihood or probability score can be assessed at different time points (e.g., before or after treatment) to monitor disease progression or to monitor treatment effectiveness (e.g., therapeutic efficacy). In still other embodiments, the likelihood or probability score can be used to make or influence a clinical decision (e.g., diagnosis of cancer, treatment selection, assessment of treatment effectiveness, etc.). For example, in one embodiment, if the likelihood or probability score exceeds a threshold, a physician can prescribe an appropriate treatment. In some embodiments, a test report can be generated to provide a patient with their test results, including, for example, a probability score that the patient has a disease state (e.g., cancer), a type of disease (e.g., a type of cancer), and/or a disease tissue of origin (e.g., a cancer tissue of origin).

### VI.A. Early Detection of Cancer

In some embodiments, the methods, tumor fraction estimate models, and/or classifiers of the present disclosure are used to detect the presence or absence of cancer in a subject suspected of having cancer. For example, a classifier (as described herein) can be used to determine a likelihood or probability score that a sample feature vector is from a subject that has cancer.

In one embodiment, a probability score of greater than or equal to 60 can indicated that the subject has cancer. In still other embodiments, a probability score greater than or equal to 65, greater than or equal to 70, greater than or equal to 75, greater than or equal to 80, greater than or equal to 85, greater than or equal to 90, or greater than or equal to 95, indicated that the subject has cancer. In other embodiments, a probability score can indicate the severity of disease. For example, a probability score of 80 may indicate a more severe form, or later stage, of cancer compared to a score below 80 (e.g., a score of 70). Similarly, an increase in the probability score over time (e.g., at a second, later time point) can indicate disease progression or a decrease in the probability score over time (e.g., at a second, later time point) can indicate successful treatment.

In another embodiment, a cancer log-odds ratio can be calculated for a test subject by taking the log of a ratio of a probability of being cancerous over a probability of being non-cancerous (i.e., one minus the probability of being cancerous), as described herein. In accordance with this embodiment, a cancer log-odds ratio greater than 1 can indicate that the subject has cancer. In still other embodiments, a cancer log-odds ratio greater than 1.2, greater than 1.3, greater than 1.4, greater than 1.5, greater than 1.7, greater than 2, greater than 2.5, greater than 3, greater than 3.5, or greater than 4, indicated that the subject has cancer. In other embodiments, a cancer log-odds ratio can indicate the severity of disease. For example, a cancer log-odds ratio greater than 2 may indicate a more severe form, or later stage, of cancer compared to a score below 2 (e.g., a score of 1). Similarly, an increase in the cancer log-odds ratio over time (e.g., at a second, later time point) can indicate disease progression or a decrease in the cancer log-odds ratio over time (e.g., at a second, later time point) can indicate successful treatment.

According to aspects of the disclosure, the methods and systems of the present disclosure can be trained to detect or classify multiple cancer indications. For example, the methods, systems and classifiers of the present disclosure can be used to detect the presence of one or more, two or more, three or more, five or more, or ten or more different types of cancer.

In some embodiments, the cancer is one or more of head and neck cancer, liver/bileduct cancer, upper GI cancer, pancreatic/gallbladder cancer; colorectal cancer, ovarian cancer, lung cancer, multiple myeloma, lymphoid neoplasms, melanoma, sarcoma, breast cancer, and uterine cancer.

The methods and systems can further quantify cancer signal present in the subject, e.g., by determining the tumor fraction prediction based on counts of methylation variants. The analytics system may chart patients diagnosed with cancer with tumor fraction prediction(s) over the course of the cancer. The analytics system may utilize this cancer cohort to generate a predictive model as a post-diagnostic tool. The quantified cancer signal may be used to predict a prognosis for the subject; staging of cancer; whether a cancer is benign, malignant, aggressive, or spreading; predicting likelihood of recurrence post-treatment; likelihood of treatment success; etc. For example, the predictive model may predict a prognosis for a subject based on the tumor fraction prediction. The prognosis may indicate a life expectancy, a likelihood of remission (partial or complete), a likelihood of recurrence, a likelihood of spreading, etc. Based on the prognosis, the analytics system may provide a list of recommended treatments. For example, a poor prognosis may recommend, among other treatments, clinical trials, chemotherapy, surgery, etc. A fair prognosis may recommend less-intensive treatment options.

### VI.B. Cancer and Treatment Monitoring

In certain embodiments, the first time point is before a cancer treatment (e.g., before a resection surgery or a therapeutic intervention), and the second time point is after a cancer treatment (e.g., after a resection surgery or therapeutic intervention), and the method utilized to monitor the effectiveness of the treatment. For example, if the second likelihood or probability score decreases compared to the first likelihood or probability score, then the treatment is considered to have been successful. However, if the second likelihood or probability score increases compared to the first likelihood or probability score (or the likelihood or probability score remains substantially the same), then the treatment is considered to have not been successful. In response to ineffective treatment options, the analytics system may recommend alternative treatment options excluding the current ineffective treatment. In other embodiments, both the first and second time points are before a cancer treatment (e.g., before a resection surgery or a therapeutic intervention). In still other embodiments, both the first and the second time points are after a cancer treatment (e.g., before a resection surgery or a therapeutic intervention) and the method is used to monitor the effectiveness of the treatment or loss of effectiveness of the treatment. In still other embodiments, cfDNA samples may be obtained from a cancer patient at a first and second time point and analyzed. e.g., to monitor cancer progression, to determine if a cancer is in remission (e.g., after treatment), to monitor or detect residual disease or recurrence of disease, or to monitor treatment (e.g., therapeutic) efficacy.

Those of skill in the art will readily appreciate that test samples can be obtained from a cancer patient over any desired set of time points and analyzed in accordance with the methods of the disclosure to monitor a cancer state in the patient. In some embodiments, the first and second time points are separated by an amount of time that ranges from about 15 minutes up to about 30 years, such as about 30 minutes, such as about 1, 2, 3, 4, 5, 6, 7, 8, 9, 10, 11, 12, 13, 14, 15, 16, 17, 18, 19, 20, 21, 22, 23, or about 24 hours, such as about 1, 2, 3, 4, 5, 10, 15, 20, 25 or about 30 days, or such as about 1, 2, 3, 4, 5, 6, 7, 8, 9, 10, 11, or 12 months, or such as about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5 or about 30 years. In other embodiments, test samples can be obtained from the patient at least once every 3 months, at least once every 6 months, at least once a year, at least once every 2 years, at least once every 3 years, at least once every 4 years, or at least once every 5 years.

For some cancer patients, a tumor can be detected early enough to be cured or controlled with long-time stable maintenance therapy (e.g., hormone replacement or deprivation therapies for estrogen- and progesterone-driven breast cancers or androgen- or testosterone-driven prostate cancers). Prior cancer patients can obtain regular surveillance to catch recurrence or increase of minimal residual disease. Patients with a prior cancer can be at above-average risk of developing a second, independent primary cancer and therefore can be advised to have regular screening and early detection for all other cancers. Surveillance of prior or actively treated cancer can be independent of screening or early detection of other cancers, which increases effort, costs, and reduces patient compliance to this long-term high-burden testing regiment.

A sample (e.g., non-cancer sample, a biopsy sample, or plasma from a blood draw) and an assay (e.g., targeted methylation) can be used to perform cancer surveillance or minimal residual disease detection together with cancer screening and early detection of secondary primary cancers. Surveillance can be performed at a higher frequency (e.g., every 6 months) than cancer screening (e.g., every year). Regular, more frequent test intervals can be used to determine a change in the signal over time. Such longitudinal data can inform cancer prognosis and therapy decisions when either recurrence of a previously treated cancer or the occurrence of a new cancer is detected.

Multi-cancer early detection can be performed for possible secondary primary cancers independent of an either tumor-informed or non-tumor-informed minimal molecular residual disease or recurrence detection for a previously known and previously treated cancer type. A tumor-informed minimal molecular residual disease or recurrence detection can use genetic or epigenetic variants determined from a tissue sample, for example a biopsy of cancerous tissue. When cancer variants from a tissue sample are known, the same plasma sample can be used with two different analysis and classification methods. One method can be a cancer screening method using a trained classifier that detects patterns indicative of the presence of cancer with no prior knowledge and possible cancer type and cancer variants. Another method can be a method for a tissue-informed residual disease or recurrence detection that focuses on variants known to occur in a cancer that the patient is diagnosed with and is treated or surveilled for.

The test for MRD or recurrence of a previously treated cancer can estimate the presence and fraction of tumor-derived fragments in the sample. The measurement can be supported by information from tumor-derived genomic or methylation variants observed on a tissue sample of that tumor. Recurrence or MRD can be reported when the estimated tumor fraction exceeds a pre-specified threshold that is established to reach a target specificity of the analysis in the presence of biological and technical noise. Consequently, a tumor fraction estimate can be above zero even for a negative result. However, when recurrence or MRD is detected in subsequent blood draws, then previous tumor fraction estimates (even if below a detection threshold) can be used to assess a gradient of tumor fraction increase over time, to assist patient prognosis and to inform treatment decisions for the recurred cancer.

Similarly, the cancer early detection test can estimate the probability that any cancer is present. A cancer detection can be reported when this probability exceeds a pre-specified threshold established to reach a target specificity of the analysis. Also for the cancer early detection test, this probability of cancer can be above zero but below a detection threshold. When cancer is detected in subsequent blood draws, then the increase of the probability of cancer of the same type over time can be determined and again inform patient prognosis and treatment decisions. For this case, the temporal derivative of the cancer signal strength (e.g., how much the signal changes over time) instead of the absolute value can be input for cancer detection and/or patient management decisions. In one or more embodiments, the cancer signal over time may include estimated tumor fractions of one or more tissues of origin (e.g., as described above in reference to FIG. 7).

In one or more embodiments of MRD detection, a plasma sample is taken for screening and early detection and provides a first reference for later treatment monitoring and MRD detection. Cancer diagnosis generally includes taking a biopsy of the cancerous tissue, and often treatment involves surgical removal of a tumor. Post treatment (for example after surgical resection), residual disease can be detected from a plasma sample, for example drawn a couple of days after the begin of therapy. The plasma sample can be shipped for processing and analysis soon after blood draw independent of tissue collection and processing, e.g., according to the method 100 of FIG. 1. The analysis of the plasma sample can start independent of the status of tissue processing. A non-tumor-informed analysis can determine if cancer signal was detected and provide a tumor burden measurement like VAF, and a confidence for this value (for example, a 95% confidence interval). If a plasma sample pre-treatment, for example from a screening or early detection test, is available, then tumor-derived fragments from this sample can be used for a first attempt with a tumor-informed analysis.

The analytics system 200 may then evaluate whether to proceed with analyses based on the detected cancer signal. The non-tumor-informed analysis or tumor-informed analysis based on a previous plasma sample might or might not detect cancer signal. If cancer signal is detected, then the use of training data to determine a measurement like VAF may yield only few informative ctDNA fragments in the plasma sample and, therefore low confidence in the quantitation. If the plasma sample contained a sufficient number of informative fragments that allow to provide clinically sufficient information on VAF, then the analytics system can return VAF with the high confidence as the test result, saving on sequencing of the tissue sample, biopsy slide review, biopsy slide mark-up, biopsy slide dissection, or some combination thereof. However, if no cancer signal detected and/or there is low confidence in VAF estimates, the analytics system may continue with tissue processing and sequencing, i.e., the analytics system continues to tumor-informed analysis. The analytics system 200 may return the VAF or tumor burden based on the tumor-informed analysis. The analytic system 200 may also return whether there was no cancer signal detected in the non-tumor-informed analysis or whether there was low confidence in the VAF estimates in the non-tumor-informed analysis.

The detection of an inflammatory process can rely on minimal residual disease detection from a targeted methylation assay. The methods described can enable detection and quantitation of the presence of tumor, abnormal, unusual, or disease-specific methylation patterns on a per-fragment basis. The cfDNA fragments with these telltale methylation patterns (e.g., using one or more of the variant models) can then be used to quantify the presence of a tumor of the target type in a blood draw from a cancer patient.

In example implementations, the presence of cfDNA fragments from apoptotic non-cancerous cells can be detected with the same assay and methodology described throughout, that are used to detect and quantify presence of cancer-derived cfDNA fragments. In addition to different tumor types, unusual or cell-type-specific methylation patterns from the following cells can be used to detect severe side effects of immune checkpoint inhibition: colon epithelial cells contributing to bowel inflammation, stomach epithelial cells contributing to bowel inflammation, pancreatic ductal cells contributing to pancreatitis, pancreatic acinar cells contributing to pancreatitis, pancreatic endocrine cells contributing to pancreatitis, hepatocytes contributing to hepatitis, and cholangiocytes contributing to hepatitis.

While immune checkpoint inhibitors are, for example, used as treatment for lung cancers, bladder cancers, and melanomas, severe side effects due to uncontrolled inflammation may occur in colon, pancreas, or liver. Consequently, the apoptotic DNA of cells shed by the tumor and from cells shed due to inflammation come from a different cell origin and can be detected and quantified independently from the same blood draw, assay, and bioinformatics pipeline. The analytics system may identify such propensity for uncontrolled inflammation in a patient based on the quantified presence of apoptotic non-cancerous cells. The analytics system and/or a physician (or other related healthcare provider) can recommend avoiding immune checkpoint inhibitors to minimize uncontrolled inflammation.

### VI.C. Treatment

In still another embodiment, information obtained from any method described herein (e.g., the likelihood or probability score) can be used to make or influence a clinical decision (e.g., diagnosis of cancer, treatment selection, assessment of treatment effectiveness, etc.). For example, in one embodiment, if the likelihood or probability score exceeds a threshold, a physician can prescribe an appropriate treatment (e.g., a resection surgery, radiation therapy, chemotherapy, and/or immunotherapy). In some embodiments, information such as a likelihood or probability score can be provided as a readout to a physician or subject.

A classifier or a model (as described herein) can be used to determine a likelihood or probability score that a sample feature vector is from a subject that has cancer. In one embodiment, an appropriate treatment (e.g., resection surgery or therapeutic) is prescribed when the likelihood or probability exceeds a threshold. For example, in one embodiment, if the likelihood or probability score is greater than or equal to 60, one or more appropriate treatments are prescribed. In another embodiments, if the likelihood or probability score is greater than or equal to 65, greater than or equal to 70, greater than or equal to 75, greater than or equal to 80, greater than or equal to 85, greater than or equal to 90, or greater than or equal to 95, one or more appropriate treatments are prescribed. In other embodiments, a cancer log-odds ratio can indicate the effectiveness of a cancer treatment. For example, an increase in the cancer log-odds ratio over time (e.g., at a second, after treatment) can indicate that the treatment was not effective. Similarly, a decrease in the cancer log-odds ratio over time (e.g., at a second, after treatment) can indicate successful treatment. In another embodiment, if the cancer log-odds ratio is greater than 1, greater than 1.5, greater than 2, greater than 2.5, greater than 3, greater than 3.5, or greater than 4, one or more appropriate treatments are prescribed.

In some embodiments, the treatment is one or more cancer therapeutic agents selected from the group including a chemotherapy agent, a targeted cancer therapy agent, a differentiating therapy agent, a hormone therapy agent, and an immunotherapy agent. For example, the treatment can be one or more chemotherapy agents selected from the group including alkylating agents, antimetabolites, anthracyclines, anti-tumor antibiotics, cytoskeletal disruptors (taxans), topoisomerase inhibitors, mitotic inhibitors, corticosteroids, kinase inhibitors, nucleotide analogs, platinum-based agents and any combination thereof. In some embodiments, the treatment is one or more targeted cancer therapy agents selected from the group including signal transduction inhibitors (e.g. tyrosine kinase and growth factor receptor inhibitors), hi stone deacetylase (HDAC) inhibitors, retinoic receptor agonists, proteosome inhibitors, angiogenesis inhibitors, and monoclonal antibody conjugates. In some embodiments, the treatment is one or more differentiating therapy agents including retinoids, such as tretinoin, alitretinoin and bexarotene. In some embodiments, the treatment is one or more hormone therapy agents selected from the group including anti-estrogens, aromatase inhibitors, progestins, estrogens, anti-androgens, and GnRH agonists or analogs. In one embodiment, the treatment is one or more immunotherapy agents selected from the group comprising monoclonal antibody therapies such as rituximab (RITUXAN) and alemtuzumab (CAMPATH), non-specific immunotherapies and adjuvants, such as BCG, interleukin-2 (IL-2), and interferon-alfa, immunomodulating drugs, for instance, thalidomide and lenalidomide (REVLIMID). It is within the capabilities of a skilled physician or oncologist to select an appropriate cancer therapeutic agent based on characteristics such as the type of tumor, cancer stage, previous exposure to cancer treatment or therapeutic agent, and other characteristics of the cancer.

### VII. KIT IMPLEMENTATION

Also disclosed herein are kits for performing the methods described above including the methods relating to the cancer classifier. The kits may include one or more collection vessels for collecting a sample from the individual comprising genetic material. The sample can include blood, plasma, serum, urine, fecal, saliva, other types of bodily fluids, or any combination thereof. Such kits can include reagents for isolating nucleic acids from the sample. The reagents can further include reagents for sequencing the nucleic acids including buffers and detection agents. In one or more embodiments, the kits may include one or more sequencing panels comprising probes for targeting particular genomic regions, particular mutations, particular genetic variants, or some combination thereof. In other embodiments, samples collected via the kit are provided to a sequencing laboratory that may use the sequencing panels to sequence the nucleic acids in the sample.

A kit can further include instructions for use of the reagents included in the kit. For example, a kit can include instructions for collecting the sample, extracting the nucleic acid from the test sample. Example instructions can be the order in which reagents are to be added, centrifugal speeds to be used to isolate nucleic acids from the test sample, how to amplify nucleic acids, how to sequence nucleic acids, or any combination thereof. The instructions may further illumine how to operate a computing device as the analytics system 200, for the purposes of performing the steps of any of the methods described.

In addition to the above components, the kit may include computer-readable storage media storing computer software for performing the various methods described throughout the disclosure. One form in which these instructions can be present is as printed information on a suitable medium or substrate, e.g., a piece or pieces of paper on which the information is printed, in the packaging of the kit, in a package insert. Yet another means would be a computer readable medium, e.g., diskette, CD, hard-drive, network data storage, on which the instructions have been stored in the form of computer code. Yet another means that can be present is a website address or QR code which can be used via the internet to access the information at a removed site.

### VIII. ADDITIONAL CONSIDERATIONS

The foregoing description of the embodiments of the disclosure has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the disclosure in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules can be embodied in software, firmware, hardware, or any combinations thereof.

Any of the steps, operations, or processes described herein can be performed or implemented with one or more hardware or software modules, alone or in combination with other devices. In some embodiments, a software module is implemented with a computer program product including a computer-readable non-transitory medium containing computer program code, which can be executed by a computer processor for performing any or all of the steps, operations, or processes described.

Embodiments can also relate to a product that is produced by a computing process described herein. Such a product can include information resulting from a computing process, where the information is stored on a non-transitory, tangible computer readable storage medium and can include any embodiment of a computer program product or other data combination described herein.

Finally, the language used in the specification has been principally selected for readability and instructional purposes, and it cannot have been selected to delineate or circumscribe the inventive subject matter. It is therefore intended that the scope of the invention be limited not by this detailed description, but rather by any claims that issue on an application based hereon. Accordingly, the disclosure of the embodiments herein is intended to be illustrative, but not limiting, of the scope of the invention, which is set forth in the following claims.

1. A computer-implemented method for generating a tumor fraction prediction from a cell-free deoxyribonucleic acid (cfDNA) sample of a subject, the computer-implemented method comprising:
receiving a dataset of methylation sequence reads from the cfDNA sample of the subject;
dividing the dataset into a plurality of variants, wherein each variant comprises a methylation pattern over one or more CpG sites;
filtering the plurality of variants based on a bank of reference sequence reads to generate a filtered subset of variants, the bank comprising reads generated from non-cancer cfDNA samples and biopsy samples of a plurality of tissues of reference individuals;
determining, for each variant in the filtered subset, a count of methylation sequence reads that include the variant;
inputting the counts of methylation sequence reads for the variants of the filtered subset to a model that is trained based on recurrence rates of the plurality of variants; and
generating, using the model, the tumor fraction prediction of the cfDNA sample.

2. The computer-implemented method of claim 1, wherein the recurrence rates of the plurality of variants are determined based on the reference sequence reads in the bank.

3. The computer-implemented method of claim 1, wherein filtering the plurality of variants based on reference sequence reads to generate the filtered subset of variants comprises filtering out one or more variants whose rates of presence in the non-cancer samples exceeds a threshold.

4. The computer-implemented method of claim 1, wherein a particular recurrence rate of a particular variant corresponds to a rate of observation of the particular variant among the reference sequence reads in the bank.

5. The computer-implemented method of claim 1, wherein the tumor fraction prediction is a distribution of probability of a fraction of fragments in the cfDNA sample that are tumor derived.

6. The computer-implemented method of claim 1, wherein the tumor fraction prediction is a fraction of fragments in the cfDNA sample that is tumor derived.

7. The computer-implemented method of claim 1, wherein the model comprises at least one probabilistic model, the probabilistic model comprising a Poisson distribution for a particular variant, and the Poisson distribution is weighted by the recurrence rate of the particular variant.

8. The computer-implemented method of claim 1, wherein the model comprises a plurality of probabilistic distributions, each probabilistic distribution corresponding to a particular variant and parameterized based on a site-specific noise rate of the particular variant and per-site sequencing depth of the particular variant.

9. The computer-implemented method of claim 8, wherein each probabilistic distribution corresponding to a particular variant is further parameterized based on at least one of: a depth of the cfDNA sample, a targeted panel pull-down efficiency of the cfDNA sample, and an estimated tumor fraction of the cfDNA sample.

10. The computer-implemented method of claim 1, wherein a count for each variant of the filtered subset comprises a count of methylation sequence reads of the cfDNA sample that include the methylation pattern over the one or more CpG sites of the variant.

11. The computer-implemented method of claim 1, wherein a particular variant that comprises a plurality of contiguous CpG sites is encoded by a series of binary values, the series corresponds to the contiguous CpG sites, a first binary value at a particular CpG site represents methylation is observed, and a second binary value at the particular CPG site represents unmethylation is observed.

12. The computer-implemented method of claim 1, wherein the tumor fraction prediction comprises a plurality of fractions for a subset of tissues.

13. The computer-implemented method of claim 12, wherein each fraction represents a percentage of fragments of the cfDNA sample that is derived from each tissue of the subset of tissues.

14. The computer-implemented method of claim 12, wherein the model is a binomial mixture model assuming independence between the variants in the filtered subset of variants.

15. The computer-implemented method of claim 14, wherein the model comprises a plurality of methylation sub-models, each methylation sub-model associated with a variant in the filtered subset and parameterized by the recurrence rates of the variant across the subset of tissues and an estimated tumor fraction, wherein each methylation sub-model is configured to calculate a likelihood of observing the count of methylation sequence reads based on the count of methylation sequence reads.

16.-29. (canceled)

30. The computer-implemented method of claim 1, wherein the model is a machine-learned model.

31. The computer-implemented method of claim 30, wherein the machine-learned model is one or more of: a constant model, a binomial model, an independent site model, a neural network model, or a Markov model.

32. The computer-implemented method of claim 30, wherein the machine-learned model is trained by:
for each reference sample in the bank including the non-cancer cfDNA samples and the biopsy samples, identifying, for each variant of the filtered variants, a count of reads that include the variant;
determining, for each variant of the filtered variants, a recurrence rate for non-cancer based on the counts of reads for the variant in the non-cancer samples;
determining, for each variant of the filtered variants, a recurrence rate for cancer based on the counts of reads for the variant in the biopsy samples; and
training the model with the recurrence rates for non-cancer and the recurrence rates for cancer, wherein the model is configured to predict a tumor fraction prediction based on counts of reads for the filtered variants in a given sample.

33. The computer-implemented method of claim 1, wherein the cfDNA sample is used to perform one or more of:
cancer surveillance for a previously diagnosed cancer; and
early cancer screening for a plurality of cancer types.

34. The computer-implemented method of claim 1, wherein the cfDNA sample of the subject is a liquid biopsy collected after beginning of treatment for cancer, the computer-implemented method further comprising:
determining a confidence score of the tumor fraction prediction based on the counts of methylation sequence reads that include the filtered subset of variants;
in response to determining that the confidence score is below a confidence threshold, sequencing a tissue sample collected after the beginning of the treatment for the cancer;
receiving a second dataset of methylation sequence reads from the tissue sample of the subject;
dividing the second dataset into a second plurality of variants;
filtering the second plurality of variants based on the bank of reference sequence reads to generate a second filtered subset of variants;
determining, for each variant in the filtered subset, a second count of methylation sequence reads that include the variant;
inputting the second counts of methylation sequence reads for the variants of the second filtered subset to the model; and
generating, using the model, a second tumor fraction prediction of the tissue sample.

35. The computer-implemented method of claim 34, further comprising:
returning the second tumor fraction prediction and the tumor fraction prediction with the confidence score.

36. The computer-implemented method of claim 1, wherein the cfDNA sample of the subject is a liquid biopsy sample collected after beginning of treatment for cancer, the computer-implemented method further comprising:
determining that the tumor fraction prediction of the liquid biopsy sample is below a threshold signal;
in response to determining that the tumor fraction prediction of the liquid biopsy sample is below a threshold signal, sequencing a tissue sample collected after the beginning of the treatment for the cancer;
receiving a second dataset of methylation sequence reads from the tissue sample of the subject;
dividing the second dataset into a second plurality of variants;
filtering the second plurality of variants based on the bank of reference sequence reads to generate a second filtered subset of variants;
determining, for each variant in the filtered subset, a second count of methylation sequence reads that include the variant;
inputting the second counts of methylation sequence reads for the variants of the second filtered subset to the model; and
generating, using the model, a second tumor fraction prediction of the tissue sample.

37.-46. (canceled)

47. The computer-implemented method of claim 1, wherein the cfDNA sample is collected from the subject after beginning of a treatment for a disease in the subject, the method further comprising:
evaluating the treatment based on the tumor fraction prediction.

48. The computer-implemented method of claim 47, wherein evaluating the treatment comprises one or more of:
determining the treatment to be effective in response to determining that the tumor fraction prediction of the cfDNA sample collected after beginning the treatment is smaller than an initial tumor fraction prediction of an initial cfDNA sample collected before beginning the treatment; and
determining the treatment to be ineffective in response to determining that the tumor fraction prediction of the cfDNA sample collected after beginning the treatment is substantially equal to or greater than an initial tumor fraction prediction of an initial cfDNA sample collected before beginning the treatment, and providing a list of alternative treatments excluding the treatment in response to determining that the treatment is ineffective.

49. (canceled)

50. (canceled)

51. A non-transitory computer readable medium configured to store computer code comprising instructions for generating a tumor fraction prediction from a cell-free deoxyribonucleic acid (cfDNA) sample of a subject, wherein the instructions, when executed by one or more processors, cause the one or more processors to:
receive a dataset of methylation sequence reads from the cfDNA sample of the subject;
divide the dataset into a plurality of variants, wherein each variant comprises a methylation pattern over one or more CpG sites;
filter the plurality of variants based on a bank of reference sequence reads to generate a filtered subset of variants, the bank comprising reads generated from non-cancer cfDNA samples and biopsy samples of a plurality of tissues of reference individuals;
determine, for each variant in the filtered subset, a count of methylation sequence reads that include the variant;
input the counts of methylation sequence reads for the variants of the filtered subset to a model that is trained based on recurrence rates of the plurality of variants; and
generate, using the model, the tumor fraction prediction of the cfDNA sample.

52. (canceled)
